(19) United States
(12) Patent Application Publication
(10) Pub. No.: US 2022/0404361 A1
(43) Pub. Date: Dec. 22, 2022

(54) COMPACT SPECTROSCOPIC ANALYZER DEVICE

(71) Applicant: Si-Ware Systems, Cairo (EG)

(72) Inventors: Yasser M. Sabry, Cairo (EG); Momen Anwar, Cairo (EG); Amr O. Ghoname, Cairo (EG); Mohamed H. Al Haron, Cairo (EG); Moustafa Mohamed, Cairo (EG); Mohamed Kilany, Cairo (EG); Mazen Erfan, Cairo (EG); Bassam Saadany, Cairo (EG); Diaa Khalil, Cairo (EG); Bassem Mortada, Cairo (EG); Erik R. Deutsch, Brookline, MA (US)

(21) Appl. No.: 17/841,559

(22) Filed: Jun. 15, 2022

Related U.S. Application Data
(60) Provisional application No. 63/211,507, filed on Jun. 16, 2021.

(51) Int. Cl. G01N 33/569; G01N 21/552; G01N 21/359

(52) U.S. Cl. CPC G01N 33/56983; G01N 21/552; G01N 21/359

(57) ABSTRACT

Aspects relate to a spectroscopic analyzer device that can be used for biological sample detection, and specifically for virus infection detection. The spectroscopic analyzer device includes a spectrometer, such as a micro-electro-mechanical systems (MEMS) based infrared spectrometer, and an artificial intelligence (AI) for screening of viral samples. In addition, the spectroscopic analyzer device includes a light source and a disposable optical component configured to receive a sample and to facilitate light interaction with the sample.

### PRIORITY CLAIM

This application claims priority to and the benefit of Provisional Application No. 63/211,507, filed in the U.S. Patent and Trademark Office on Jun. 16, 2021, the entire content of which is incorporated herein by reference as if fully set forth below in its entirety and for all applicable purposes.

### TECHNICAL FIELD

The technology discussed below relates generally to a spectroscopic analyzer device for biological sample detection, and in particular to mechanisms for virus infection detection.

### BACKGROUND

Infrared spectroscopy provides characterization of the vibrational and rotational energy levels of molecules in different materials. When the material is exposed to infrared light, absorption of photons occurs at certain wavelengths due to transitions between vibrational levels. Today, spectrometer instruments can be found in labs and industrial environments for material identification and/or quantification in different application areas. Various topologies for spectrometry instrumentation exist, including Fourier Transform Infrared (FT-IR).

Infrared spectroscopy is a fast and low-cost mechanism for diagnosing biological samples, in general, and viral infections, specifically. Each virus has a unique molecular structure. Each of these molecular structure components has its own spectral absorption signal in the infrared range, showing stronger absorption in the fingerprint mid-infrared region. The spectral absorption signal in the mid-infrared range is stronger since this is the fundamental region, while the signals in the near-infrared region (e.g., 7400 cm⁻¹ to 4000 cm⁻¹) are overtones and combinations of the fundamental ones. The mid-infrared spectrum at the fingerprint region are the bands corresponding to the main biomarker fragments. Based on this mechanism, various infrared absorption-based mechanisms for viral infection detection may be utilized.

For instance, near-infrared Raman spectroscopy has been used to spectrally differentiate between healthy human blood serum and blood serum with hepatitis C contamination in vitro. In addition, near-infrared spectroscopy has also been used to discriminate influenza virus-infected nasal fluids and to diagnose HIV-1 infection. Furthermore, the detection of malaria parasites in dried human blood spots using mid-infrared spectroscopy and regression analysis has been reported.

Near-infrared spectroscopy has also been used to detect viruses in animals, insects and plants. For instance, near-infrared spectroscopy has been used as a rapid, reagent-free, and cost-effective tool to noninvasively detect ZIKV in heads and thoraces of intact Aedes aegypti mosquitoes with prediction accuracies of 94.2% to 99.3% relative to polymerase chain reaction (PCR). In addition, near-infrared spectroscopy and aquaphotomics have been used as an approach for rapid in vivo diagnosis of virus infected soybean. Detection and quantification of poliovirus infection using FTIR spectroscopy in cell cultures have also been reported.

### SUMMARY

The following presents a summary of one or more aspects of the present disclosure, in order to provide a basic understanding of such aspects. This summary is not an extensive overview of all contemplated features of the disclosure, and is intended neither to identify key or critical elements of all aspects of the disclosure nor to delineate the scope of any or all aspects of the disclosure. Its sole purpose is to present some concepts of one or more aspects of the disclosure in a form as a prelude to the more detailed description that is presented later.

In an example, a spectroscopic analyzer device is disclosed. The spectroscopic analyzer device includes a light source configured to produce incident light, a disposable optical component configured to receive a sample and further configured to receive input light corresponding to the incident light or an interference beam produced based on the incident light, the disposable optical component further configured to produce output light based on light interaction with the sample. The spectroscopic analyzer device further includes a spectrometer configured to receive the incident light from the light source or the output light from the disposable optical component and further configured to produce the interference beam, the interference beam corresponding to the input light or being produced based on the output light. The spectroscopic analyzer device further including a detector configured to obtain a spectrum of the sample based on the interference beam or the output light, and an artificial intelligence (AI) engine configured to receive the spectrum and to generate a result indicative of at least one parameter associated with the sample.

These and other aspects of the invention will become more fully understood upon a review of the detailed description, which follows. Other aspects, features, and embodiments of the present invention will become apparent to those of ordinary skill in the art, upon reviewing the following description of specific, exemplary embodiments of the present invention in conjunction with the accompanying figures. While features of the present invention may be discussed relative to certain embodiments and figures below, all embodiments of the present invention can include one or more of the advantageous features discussed herein. In other words, while one or more embodiments may be discussed as having certain advantageous features, one or more of such features may also be used in accordance with the various embodiments of the invention discussed herein. In similar fashion, while exemplary embodiments may be discussed below as device, system, or method embodiments it should be understood that such exemplary embodiments can be implemented in various devices, systems, and methods.

### BRIEF DESCRIPTION OF THE DRAWINGS

FIG. 1 is a diagram illustrating a spectrometer according to some aspects.

FIG. 2 illustrates an example of a workflow for building an AI engine according to some aspects.

FIG. 3 is a diagram illustrating an example of a spectroscopic analyzer device according to some aspects.

FIG. 4 is a flow diagram illustrating an exemplary process flow of sample analysis using the spectroscopic analyzer device according to some aspects.

FIG. 5 is a diagram illustrating an example of detection of the presence of a virus using a spectrometer according to some aspects.

FIG. 6 is a diagram illustrating an example of AI engine training according to some aspects.

FIG. 7 is a diagram illustrating an example of a model for predicting values based on reference values according to some aspects.

FIGS. 8A and 8B are diagrams illustrating examples of a model for clustering/classifying values according to some aspects.

FIG. 9 is a diagram illustrating an example of a spectroscopic analyzer device including a multi-pass cell according to some aspects.

FIG. 10 is a diagram illustrating another example of a spectroscopic analyzer device including a multi-pass cell according to some aspects.

FIGS. 11A and 11B illustrate an example of a spectroscopic analyzer device including an ATR element according to some aspects.

FIGS. 12A and 12B are diagrams illustrating other examples of spectroscopic analyzer devices including an ATR element according to some aspects.

FIGS. 13A, 13B, and 13C are diagrams illustrating other examples of spectroscopic analyzer devices including an ATR element according to some aspects.

FIGS. 14A and 14B are diagrams illustrating other examples of spectroscopic analyzer devices including an ATR element according to some aspects.

FIGS. 15A and 15B are diagrams illustrating examples of a spectroscopic analyzer device including a spectrometer in the light path between the light source and ATR element according to some examples.

FIG. 16 is a diagram illustrating another example of a spectroscopic analyzer device including an ATR element according to some aspects.

FIGS. 17A and 17B are diagrams illustrating another example of a spectroscopic analyzer device including an ATR element according to some aspects.

FIG. 18 is a diagram illustrating another example of a spectroscopic analyzer device including an ATR element according to some aspects.

FIG. 19 is a diagram illustrating another example of a spectroscopic analyzer device including an ATR element according to some aspects.

FIGS. 20A and 20B are diagrams illustrating another example of a spectroscopic analyzer device including an ATR element according to some aspects.

FIGS. 21A and 21B are diagrams illustrating another example of a spectroscopic analyzer device including an ATR element according to some aspects.

FIGS. 22A and 22B are diagrams illustrating an example of a functionalized ATR element according to some aspects.

FIG. 23 is a diagram illustrating an example of the effect of different excitations on the measured spectrum.

FIG. 24 is a diagram illustrating an example technique for amplifying the signal of a sample according to some aspects.

FIGS. 25A and 25B are diagrams illustrating an example of another technique for optical signal enhancement of a sample according to some aspects.

FIGS. 26A and 26B are diagrams illustrating examples of pre-concentration of a sample according to some aspects.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Various aspects of the disclosure relate to a spectroscopic analyzer device that combines a micro-electro-mechanical systems (MEMS) based IR spectrometer and artificial intelligence (AI) for screening of viral samples. Spectroscopy is the study of physical and/or chemical properties of materials by analyzing their response to light. In a MEMS spectrometer, all of the optical and mechanical components are integrated on a single MEMS chip, enabling FTIR functionality on a chip scale.

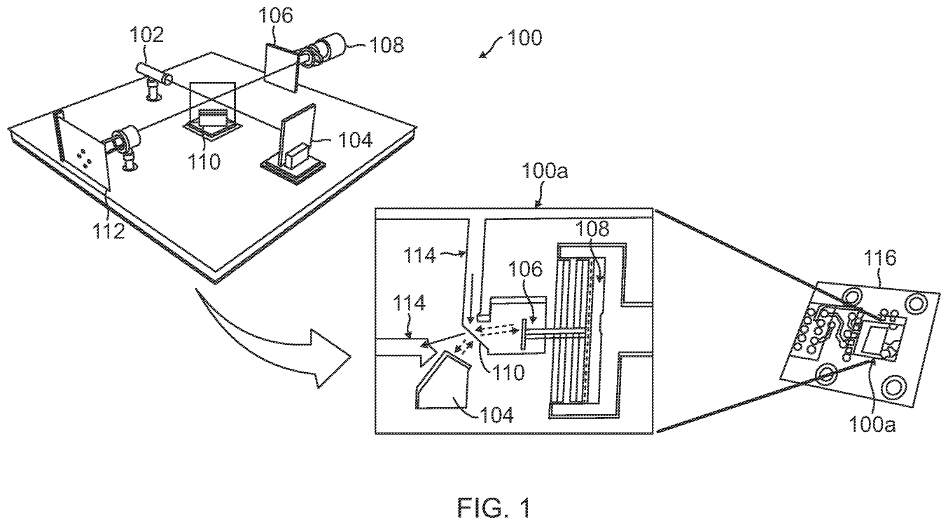

FIG. 1 is a diagram illustrating a spectrometer 100 according to some aspects. The spectrometer 100 may be, for example, a Fourier Transform infrared (FTIR) spectrometer. In the example shown in FIG. 1, the spectrometer 100 is a Michelson FTIR interferometer. In other examples, the spectrometer may include an FTIR Fabry-Perot interferometer.

FTIR spectrometers measure a single-beam spectrum (power spectral density (PSD)), where the intensity of the single-beam spectrum is proportional to the power of the radiation reaching the detector. In order to measure the absorbance of a sample, the background spectrum (i.e., the single-beam spectrum in absence of a sample) may first be measured to compensate for the instrument transfer function. The single-beam spectrum of light transmitted or reflected from the sample may then be measured. The absorbance of the sample may be calculated from the transmittance, reflectance, or trans-reflectance of the sample. For example, the absorbance of the sample may be calculated as the ratio of the spectrum of transmitted light, reflected light, or trans-reflected light from the sample to the background spectrum.

The interferometer 100 includes a fixed mirror 104, a moveable mirror 106, a beam splitter 110, and a detector 112 (e.g., a photodetector). A light source 102 associated with the spectrometer 100 is configured to emit an input beam and to direct the input beam towards the beam splitter 110. The light source 102 may include, for example, a laser source, one or more wideband thermal radiation sources, or a quantum source with an array of light emitting devices that cover the wavelength range of interest.

The beam splitter 110 is configured to split the input beam into two beams. One beam is reflected off of the fixed mirror 104 back towards the beam splitter 110, while the other beam is reflected off of the moveable mirror 106 back towards the beam splitter 110. The moveable mirror 106 may be coupled to an actuator 108 to displace the movable mirror 106 to the desired position for reflection of the beam. An optical path length difference (OPD) is then created between the reflected beams that is substantially equal to twice the mirror 106 displacement. In some examples, the actuator 108 may include a micro-electro-mechanical systems (MEMS) actuator, a thermal actuator, or other type of actuator.

The reflected beams interfere at the beam splitter 110 to produce an output light beam, allowing the temporal coherence of the light to be measured at each different Optical Path Difference (OPD) offered by the moveable mirror 106. The signal corresponding to the output light beam may be detected and measured by the detector 112 at many discrete positions of the moveable mirror 106 to produce an interferogram. In some examples, the detector 112 may include a detector array or a single pixel detector. The interferogram data verses the OPD may then be input to a processor (not shown, for simplicity). The spectrum may then be retrieved, for example, using a Fourier transform carried out by the processor.

In some examples, the interferometer 100 may be implemented as a MEMS interferometer 100a (e.g., a MEMS chip). The MEMS chip 100a may then be attached to a printed circuit board (PCB) 116 that may include, for example, one or more processors, memory devices, buses, and/or other components. In some examples, the PCB 116 may include a spectrum analyzer, such as an AI engine, configured to receive and process the spectrum. As used herein, the term MEMS refers to the integration of mechanical elements, sensors, actuators and electronics on a common silicon substrate through microfabrication technology. For example, the microelectronics are typically fabricated using an integrated circuit (IC) process, while the micromechanical components are fabricated using compatible micromachining processes that selectively etch away parts of the silicon wafer or add new structural layers to form the mechanical and electromechanical components. One example of a MEMS element is a micro-optical component having a dielectric or metallized surface working in a reflection or refraction mode. Other examples of MEMS elements include actuators, detector grooves and fiber grooves.

In the example shown in FIG. 1, the MEMS interferometer 100a may include the fixed mirror 104, moveable mirror 106, beam splitter 110, and MEMS actuator 108 for controlling the moveable mirror 106. In addition, the MEMS interferometer 100a may include fibers 114 for directing the input beam towards the beam splitter 110 and the output beam from the beam splitter 110 towards the detector (e.g., detector 112). In some examples, the MEMS interferometer 100a may be fabricated using a Deep Reactive Ion Etching (DRIE) process on a Silicon On Insulator (SOI) wafer in order to produce the micro-optical components and other MEMS elements that are able to process free-space optical beams propagating parallel to the SOI substrate. For example, the electro-mechanical designs may be printed on masks and the masks may be used to pattern the design over the silicon or SOI wafer by photolithography. The patterns may then be etched (e.g., by DRIE) using batch processes, and the resulting chips (e.g., MEMS chip 100a) may be diced and packaged (e.g., attached to the PCB 116).

For example, the beam splitter 110 may be a silicon/air interface beam splitter (e.g., a half-plane beam splitter) positioned at an angle (e.g., 45 degrees) from the input beam. The input beam may then be split into two beams L1 and L2, where L1 propagates in air towards the moveable mirror 106 and L2 propagates in silicon towards the fixed mirror 104. Here, L1 originates from the partial reflection of the input beam from the half-plane beam splitter 110, and thus has a reflection angle equal to the beam incidence angle. L2 originates from the partial transmission of the input beam through the half-plane beam splitter 110 and propagates in silicon at an angle determined by Snell's Law. In some examples, the fixed and moveable mirrors 104 and 106 are metallic mirrors, where selective metallization (e.g., using a shadow mask during a metallization step) is used to protect the beam splitter 110. In other examples, the mirrors 104 and 106 are vertical Bragg mirrors that can be realized using, for example, DRIE.

In some examples, the MEMS actuator 108 may be an electrostatic actuator formed of a comb drive and spring. For example, by applying a voltage to the comb drive, a potential difference results across the actuator 108, which induces a capacitance therein, causing a driving force to be generated as well as a restoring force from the spring, thereby causing a displacement of moveable mirror 106 to the desired position for reflection of the beam back towards the beam splitter 110.

The unique information from the vibrational absorption bands of a molecule are reflected in an infrared spectrum that may be produced, for example, by the spectrometer 100 shown in FIG. 1. By applying spectral numerical processing and statistical analysis to a spectrum, the information in the spectrum may be identified or otherwise classified. The application of statistical methods to the analysis of experimental data is traditionally known as chemometrics, and more recently as artificial intelligence.

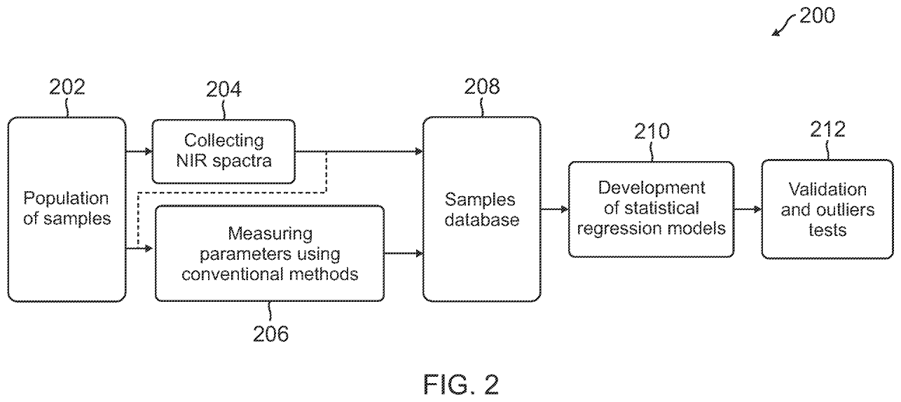

FIG. 2 illustrates an example of a workflow 200 for building an AI engine according to some aspects. To begin building the AI engine, a group or population of samples 202 is obtained for measurements by a spectrometer, such as the spectrometer 100 shown in FIG. 1, to produce spectra 204. At the same time, these samples 202 can also be measured by conventional methods and the values recorded as reference values 206. These reference values 206 together with the spectra 204 form a samples database 208 that is used to teach the AI engine (e.g., machine learning) how to interpret the spectra and transform the spectra to certain values (e.g., results). For example, the samples database 208 may be used in the development of statistical regression models (e.g., calibration models) 210 that may then be applied to a spectrum of a sample to produce a result (e.g., a positive or negative test result or antibody level) associated with the sample. Validation and outliers detection 212 of the test results may then be performed to refine the calibration model(s).

Since the spectrum produced by infrared (IR) spectroscopy are instantaneous, unlike conventional analysis methods, there is no need to wait for certain transformations (e.g., chemical transformations) to occur within the sample. Different physical and chemical parameters of the sample can be analyzed with a single scan.

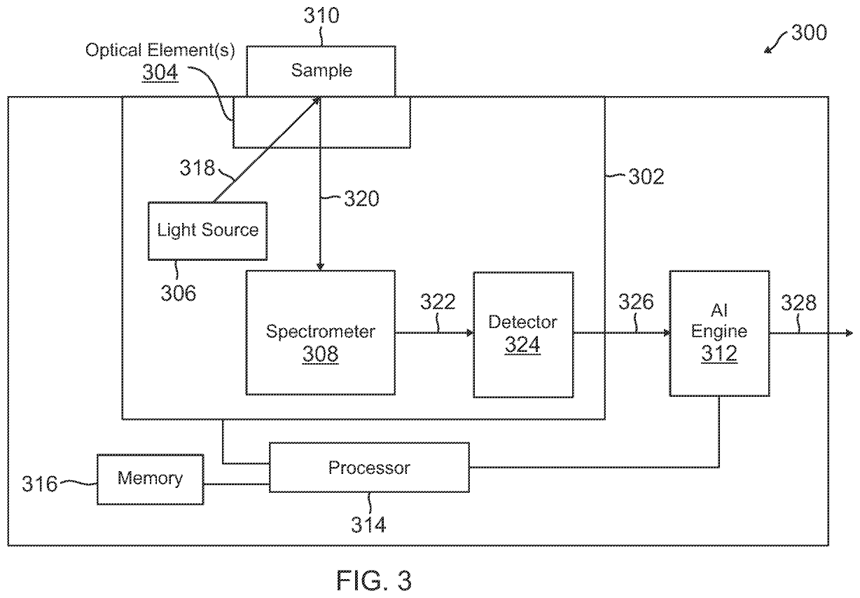

FIG. 3 is a diagram illustrating an example of a spectral analyzer device 300 according to some aspects. The spectral analyzer device 300 can be an efficient tool to contain the spread of the infection in a pandemic situation, for example COVID-19, and can facilitate the mobility of decision makers that may decide whether to provide or prevent access to a facility by various test subjects (e.g., human, animal, or plant subjects). The device 300 is compact, portable, and cheap and can be used for a mass screening campaign or screening for providing access to a certain facility or passing a gate. The analysis is ultra-rapid and very low cost. This includes work, entertainment, social, and residential facilities in addition to others.

The spectral analyzer device 300 includes an optical measurement device 302, an artificial intelligence (AI) engine 312, a processor 314, and a memory 316. The processor 314 may include a single processing device or a plurality of processing devices. Such a processing device may be a microprocessor, micro-controller, digital signal processor, microcomputer, central processing unit, field programmable gate array, programmable logic device, logic circuitry, analog circuitry, digital circuitry, and/or any device that manipulates signals (analog and/or digital) based on hard coding of the circuitry and/or operational instructions. The memory 316 may be a single memory device, a plurality of memory devices, and/or embedded circuitry of the processor 314. Such a memory device may be a read-only memory, random access memory, volatile memory, non-volatile memory, static memory, dynamic memory, flash memory, cache memory, and/or any device that stores digital information, including instructions (e.g., code) that may be executed by the processor 314.

The optical measurement device 302 includes at least one light source 306, one or more optical elements 304, a spectrometer 308, and a detector 324. The spectrometer 308 may include, for example, a diffraction element, a Michelson interferometer, a Fabry-Perot cavity, a spatial light modulator, or a birefringent device. In some examples, the spectrometer 308 includes a MEMS interference device, such as the MEMS FTIR based spectrometer, as shown in FIG. 1. The MEMS interferometer enables generating a spectrum in millisecond time scale since the moving micromirror is driven by a MEMS actuator. The light source(s) 306 may include, for example, a laser source or wideband source. In some examples, the light source(s) 306 may be infrared or near-infrared light source(s).

The optical element(s) 306 may include a disposable optical component configured to receive a sample 310. The light source(s) 306 can be configured to generate incident light 318 and to direct the incident light 318 towards the sample 310 via the optical element(s) 304 in a transmission or reflection mode. In some examples, the reflection can be in the form of external or total internal reflection. In some examples, as shown in FIG. 3, the incident light 318 from the light source 306 is received by the optical element(s) 304 as input light. The optical element(s) 304 is configured to produce output light 320 based on light interaction with the sample 310. In some examples, the optical element(s) 304 may include an attenuated total internal reflection (ATR) element (e.g., an ATR crystal) or a multi-pass cell including a plurality of reflecting elements. In some examples, the disposable optical component may correspond to the ATR element or one of the reflecting elements of the multi-pass cell. In other examples, the disposable optical component of the optical element(s) 304 may include a transparent optical window, filter, thin film crystal, or cover slip (e.g., which may be functionalized).

The output light 320 may then be input to the spectrometer 308, which is configured to produce an interference beam 322 corresponding to the output light 320. The interference beam 322 may be received by the detector 324, which may be configured to obtain a spectrum 326 of the sample 310 based on the interference beam 322.

In other examples (not shown in FIG. 3), the incident light 318 from the light source 306 may first be input to the spectrometer 308. In this example, the interference beam produced by the spectrometer 308 may be directed to the optical element(s) 304 as the input light that interacts with the sample 310 to produce the output light 320. The output light 320 may then be detected by the detector 324 to obtain the spectrum 326 of the sample 310.

The spectrum 326 may be input to the AI engine 312 for analysis and processing. The AI engine 312 is configured to process the spectrum 326 to generate a result 328 indicative of at least one parameter associated with the sample 310 from the spectrum 326. For example, the AI engine 312 may include one or more processors for processing the spectrum 326 and a memory configured to store one or more calibration models utilized by the processor in processing the spectrum. The AI engine 312 can include, for example, one or more calibration models, each built for a respective type of media (e.g., sampling method) and for a respective type of analyte under test. Examples of sampling methods include, but are not limited to, a filter or compartment for collecting the sample (e.g., breath of a patient), nasal swabs, oral swabs, a blood collection tool, or a human waste collection tool.

In some examples, a calibration model building methodology may be implemented, so that a reference technique, such as polymerase chain reaction, is used to provide reference values. Clinical data may also be complemented with laboratory data to improve the accuracy of the results in terms of sensitivity, specificity and quantifications. In some examples, a sufficient number of negative and positive samples for a particular media and a particular analyte may be used to train the corresponding calibration model. The training samples may be handled in the same way the test samples are handled. The calibration model can further be built based on a certain number of units of the spectroscopic analyzer device 300 that covers the different conditions of the device and manufacturing variations to obtain a global calibration model. In addition, the developed calibration model can be adapted for any new units produced by techniques of model transfer.

In an example operation, the processor 314 can be configured to control the spectrometer 308 and the light source(s) 306 to initiate a measurement of a sample 310. For example, the processor 314 can control the light source(s) 306 to generate and direct the incident light 318 to the sample 310. The processor 314 can further be configured to control the spectrometer 308 and detector 324 to transmit the spectrum 326 to the AI engine 312. In some examples, the calibration model in the AI engine 312 can analyze the spectrum 326 and produce a result (e.g., a value) representing the analyte under test in the form of a positive decision indicating the existence of the analyte under test or a negative decision indicating the absence of the analyte under test. The degree of positivity (e.g., infection load and severity) can also be produced by the calibration mode in the form of low, medium, and high. As another example, the result 328 may be an antibody level for a particular type of infection. In some examples, the spectrum 326 includes a measured absorption spectra and the AI engine 312 is configured to detect one or more analytes from absorption signals of the measured absorption spectra in the near-infrared frequency range. In some examples, absorption signals in the near-infrared region (frequency range) can be used to detect the analyte based on overtones and combinations of the fundamental vibrational modes. In addition, in the near-infrared region, sample preparation may not be required.

In some examples, the spectroscopic analyzer device 300 may be configured to analyze the air in the environment to detect suspended virus particles in addition to the use of various sampling methods for the detection of infected subjects (humans, animals, surfaces, etc.). In some examples, the light can interact with the air in the environment (sample) without a need for a special sampling mechanism. For example, the spectroscopic analyzer device 300 can be integrated as a part of the ventilation system of a building, room, car, etc. In an example, the AI engine 312 can detect the presence of the virus in the environment and trigger an alarm signal to initiate an action, such as evacuation, disinfection of the environment, etc.

In some examples, the processor 314 may be configured to control the spectrometer 308 to perform multiple scans (e.g., multiple measurements) of the sample 310. The spectrometer 308 or the AI engine 312 may then be configured to average the multiple measurements (e.g., multiple interferograms or multiple spectrums) to improve the sensitivity of the result 328 produced by the AI engine 312. In some examples, the result 328 may be utilized as a decision-making mechanism or to trigger an action allowing or preventing mobility of a subject (e.g., authorize or prevent access of the tested subject to a facility or through a gate).

In some examples, the sample 310 may be taken from a subject (e.g., human, animal, plant, etc.) and either applied directly to the disposable optical component or either transferred to a substrate or transport media, such as a viral transport media or other transport media (e.g., saline, phosphate buffer saline, minimum essential media, inactivation transport medium, etc.) or mixed with platform chemicals or dried used to improve sensitivity and selectivity, and then applied to the disposable optical component.

The sample 310 may be collected, for example, from exhaled breath, coughing of the subject on a substrate, or collection of body fluidics, such as saliva, nasal swabs, oral swabs, blood, body waste, non-invasive through the skin and others. The samples can be processed sample by sample or in batch mode for faster analysis especially when sample pre-analysis processing (e.g., drying or mixing with a chemical) is carried out before the spectroscopic analysis.

For analyzing biomarkers of disease or infection from a subject's breath, breath samples can be collected and contained, as discussed above. For example, the subject can provide one or more breath samples by exhaling through a disposable breath sampler. A disposable mouthpiece can be also used to allow the flow in only one way to prevent contamination. The breath sampler can be used as is to conduct the measurements or a syringe may be used to transfer the sample from the sampler into an inlet of the infrared spectroscopic analyzer device.

Alternatively, a blood sample can be collected from the subject. A small volume is needed since the sample is analyzed using an infrared spectrometer of compact size. The detection of the infectious diseases can be from the traces of the virus in the blood or the antibodies formed in the blood or biomarkers such as the combination of d-dimer protein fragments, substance P and others. Another test is to confirm the vaccination of a subject or an immunity that has been formed due to catching the diseases in the past. Once the blood sample is collected, it can be applied on the spectroscopic analyzer device directly or transferred to a cover slip then applied on the device.

A combination of one or more of the sampling and analysis methods can be used simultaneously. This is to improve the accuracy of the quantification and the sensitivity and specificity to the classification of the infection. Moreover, gas samples may be analyzed using transmission infrared spectroscopy, while dry or aqueous samples can be measured using transmission or attenuated total internal reflection spectroscopy or reflection infrared or Raman spectroscopy.

As indicated above, the collected sample can be analyzed in the near-infrared as well as the mid-infrared spectral ranges working on the fundamental vibrational lines in addition to the overtones and combination bands for the detection of the low viral load with high sensitivity. A broad spectral range also enables good differentiation with respect to other diseases, such as influenza, leading to high specificity of the test.

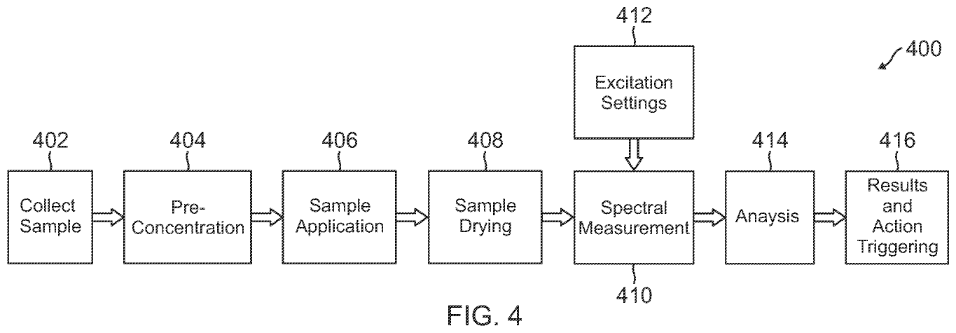

FIG. 4 is a flow diagram illustrating an exemplary process flow 400 of sample analysis using the spectroscopic analyzer device according to some aspects. At 402, a sample may be collected. At 404, pre-concentration of the sample may be performed. At 406, the sample may be applied to the spectroscopic analyzer device (e.g., the disposable optical component of the spectroscopic analyzer device). At 408, the sample may optionally be dried. In some examples, the temperature of the sample may further be sensed. At 410, a spectral measurement of the sample may be performed by the spectroscopic analyzer device to obtain a spectrum of the sample. In some examples, at 412, multiple spectra of the sample may be obtained under different excitation conditions. At 414, each of the obtained spectra may be input to the AI engine for spectral analysis. At 416, the AI engine may produce a result indicative of at least one parameter associated with the sample. In addition, the result may trigger one or more actions (e.g., provide or prevent access by a test subject to a facility). For example, the AI engine may detect a bacterial infection, a parasite infection, or their antibodies. As another example, the AI engine may detect the body biomarkers associated with a certain disease or type of infection.

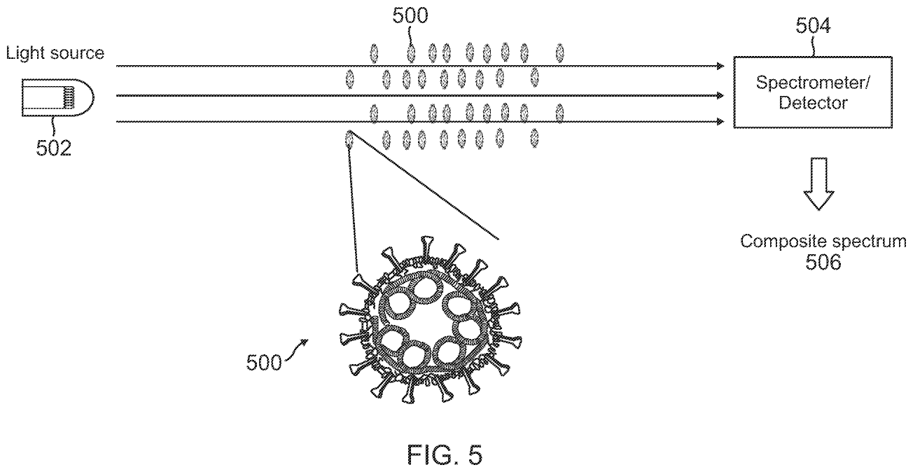

FIG. 5 is a diagram illustrating an example of detection of the presence of a virus using a spectrometer according to some aspects. In the example shown in FIG. 5, a light source 502 radiates incident light on a sample (e.g., a biological entity) 500. The sample interacts with the incident light (e.g., using one or more optical elements) and a spectrometer/detector 504 is used to detect a composite spectrum 506 of the biological entity 500. The composite spectrum 506 may contain all of the features of the biological entity 500, such as the RNA genome, spike protein, and lipid layer superimposed and overlapped. An AI engine (not shown) may then process the spectrum to recognize the original individual spectra of each of the features to detect the presence of the biological entity 500 (e.g., a virus). In addition, the AI engine may further be configured to quantify the amount of virus based on the intensity of the light versus wave number. In general, any biological species can be detected in addition to biomarkers of different diseases.

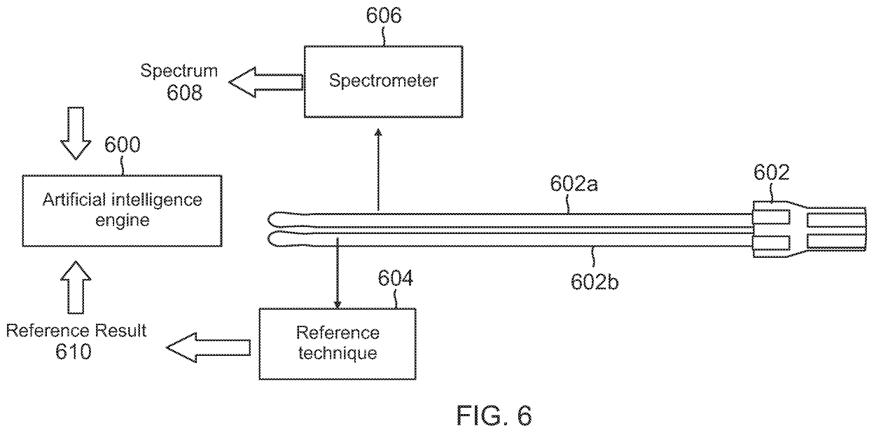

FIG. 6 is a diagram illustrating an example of AI engine training according to some aspects. Prior to the use of the spectroscopic analyzer device for the analysis of subjects (real testing), the AI engine 600 has to be trained. In the training phase, a twin sample collection device 602 may be used to collect samples from a group of subjects. In some examples, as shown in FIG. 6, the twin sample collection device 602 may include a collection of swabs. In other examples, the twin sample collection device 602 may include a collection of filters with pores size small enough to capture the sample (e.g., via coughing or breathing).

The twin sample collection device 602 is split into two parts 602a and 602b. A first part 602a is configured to enable spectroscopic measurement of a sample using, for example, a spectrometer 606 of the spectroscopic analyzer device. For example, the first part 602a may be configured to apply the sample to the disposable optical component of the spectroscopic analyzer device for measurement by the spectrometer 606 to produce a spectrum 608 of the sample.

A second part 602b is configured to enable referencing of the sample to obtain a reference result 610. For example, the second part 602b may be configured to subject the sample to analysis using a reference technique 604, such as Polymerase chain reaction (PCR) or cell culturing, to produce the reference result 610. Both the reference result 610 and the spectrum 608 may be input to the AI engine 600 to train the AI engine 600. This process is repeated for each subject in the group of subjects. In some examples, the group of subjects may include a large number of subjects statistically representing the population with positive and negative results.

In some examples, the swab corresponding to the first part 602a may be applied directly on the disposable optical component or may first be placed in a media. In case of using an aqueous media, building the calibration artificial intelligence model is doable by analysis of an amount of the aqueous media on the PCR technique 604 while another amount from the same vial tube media is measured using the spectrometer 606. However, applying the swab directly on the spectroscopic analyzer device results in less aqueous solution on the device that may need drying before conducting the spectroscopic measurement. In this case, training the AI engine 600 may involve applying one of the swabs (e.g., first part 602a) on the spectroscopic analyzer device directly while the second swab (e.g., second part 602b) is first put in a transport media in a vial and then an amount of the media is used to conduct the reference PCR test 604.

Various media can be used to transport the sample taken from the subject to the spectroscopic analyzer device. As discussed above, a calibration model can be built for each type of media. For example, a calibration model can be built for ulbecco's modified eagle medium (DMEM) cultivation media, another calibration model can be built for viral transport media (VTM), a third model can be built for Saline, a fourth model can be built for phosphate buffer saline (PBS), a fifth model can be built for the universal transport media (UTM) and so on. A universal model can then be built by factoring in the media spectrum by taking it as a background or providing it as an input to the AI engine 600.

Since the clinical samples collected from patients and used to train the AI engine 600 may not be sufficient to cover the whole range without increasing the number of the training subjects excessively, the AI engine database can be complemented with samples prepared in the laboratory with known virus concentration. In this case, cells can be maintained in media supplemented with other constitutes such as penicillin, streptomycin and fetal bovine serum, for example. All cells may be at a controlled temperature and gas. Virus stock can be obtained by inoculation. Supernatant of the infected cells can be harvested and centrifuged to remove cell debris. In addition, high concentrations of the virus samples can be achieved using centrifugal ultrafiltration process. Filters for protein purification and virus concentration can also be used. For example, water may be forced through a semipermeable membrane, while the suspended virus remains on one side of the membrane. For the titration of concentrated virus, plaque infectivity assay can be carried out. The sample is then transformed into a state similar to the sample collection methodologies discussed before. For example, transferred to a filter, a gas sample, a cover slip, a crystal or an aqueous media.

The raw measurements (e.g., spectrum 608) may be pre-processed before providing the measurements to the chemometrics model/AI engine 600. This data processing may be performed to combat the effects of light scattering and variations in the thickness of samples, to reduce the effect of the unstable information from the spectrum that arise from the instrument stability performance, and to use the most relevant portion from the spectrum in the model. For example, three different preprocessing techniques are applied to the raw samples and illustrated as below:

First, centering and scaling the individual spectra of each measured sample may be performed to reduce the scattering effect. The mean of each individual sample can be calculated and subtracted from the sample. The sample may then be scaled by its own standard deviation.

$${x}_{ik} = \frac{{x}_{ik} - {μ}_{i}}{{σ}_{i}} , \qquad ( Equation 1 )$$

where xᵢₖ is the spectral measurement for the i^{th} measured sample at the k^{th} wavenumber, and μᵢ and σᵢ are the mean and standard deviation of the i^{th} measured sample, respectively. Considering matrix X ∈ R^{N×K} consists of N measured samples with k spectral measurements each, μ ∈ R^{N×1} and σ ∈ R^{N×1} contain the N calculated means and standard deviations:

$$μ = [ \begin{matrix}{μ}_{1} \\ {μ}_{2} \\ ⋮ \\ {μ}_{N - 1} \\ {μ}_{N}\end{matrix} ] , σ = [ \begin{matrix}{σ}_{1} \\ {σ}_{2} \\ ⋮ \\ {σ}_{N - 1} \\ {σ}_{N}\end{matrix} ] \qquad ( Equation 2 )$$

Accordingly, the result spectrum X_{SNV} ∈ R^{N×1} after the pre-processing is:

$${X}_{SNV} = [ \begin{matrix}\frac{{x}_{11} - {μ}_{1}}{{σ}_{1}} & … & \frac{{x}_{1 k} - {μ}_{1}}{{σ}_{1}} \\ ⋮ & ⋱ & … \\ \frac{{x}_{N 1} - {μ}_{N}}{{σ}_{N}} & … & \frac{{x}_{NK} - {μ}_{N}}{{σ}_{N}}\end{matrix} ] \qquad ( Equation 3 )$$

Second, denoising and derivatives of the data, such as the first, second, third . . . derivative of the spectrum, may be calculated and applied to the model to enhance the results of the prediction. The main target of using the derivative is to enhance the absorption peaks information and understand the main components of the spectrum. It should be noted that the spectrum consists of discrete equally spaced measurements. Then filtering is applied based on centering a window at a certain wavelength and fitting a low order polynomial to the data points using least squares, and then the derivative is estimated from the derivative of the fitted curve. The size of the window used and the type of the fitting curve control the tradeoff between the noise reduction and curve distortion.

Third, a principal component analysis (PCA) technique may be used in both data pre-processing and the chemometrics model to achieve two different targets. The goal during preprocessing is to act as an unsupervised technique that allows data visualization and to help in understanding the classification of the samples. Data visualization becomes applicable using PCA by modeling N samples, each with K spectral variables in terms of smaller numbers of newly constructed variables zⱼ ∈ R^{N×1}, where j=1, 2, . . . , number of selected PCs. The PCs scores consist of linear combinations of the original K variables (wavenumbers) weighted by loading vectors pⱼ ∈R^{K×1}:

zⱼ=X*pⱼ (Equation 4)

where X is N×K matrix with each row representing the spectral data of each of the measured samples, the calculation of loading vectors pⱼ and the PCs are discussed more in the chemometrics model design. It is important to know that each of the original K spectral variables contributes in each of the new components accordingly a large portion of the variability on the original spectrum can be described using a smaller number of variables and the first PCs become enough to represent the variations.

A logistic regression model acts as a supervised classification model and is based on principal component regression. The main target of using PCA in the chemometric model is to compress the data from k spectral measurements to new variables with less dimensions while preserving most of the total variation in the data and removing the unrelated measured data. The procedure of PCA can be summarized as follows:

1) Apply mean centering and scaling of each feature (variable). For matrix X ∈ R^{N×K} consists of N measured samples with k spectral measurements each, each spectral measurement (variable) is mean centered and scaled by calculating the mean and standard deviation of each variable (wavenumber) as follows:

$$\underline{μ} = [ \begin{matrix}{\underline{μ}}_{1} \\ {\underline{μ}}_{2} \\ ⋮ \\ {\underline{μ}}_{K - 1} \\ {\underline{μ}}_{K}\end{matrix} ] , \underline{σ} = [ \begin{matrix}{\underline{σ}}_{1} \\ {\underline{σ}}_{2} \\ ⋮ \\ {\underline{σ}}_{K - 1} \\ {\underline{σ}}_{K}\end{matrix} ] \qquad ( Equation 5 )$$

$$\underline{X} = [ \begin{matrix}\frac{{x}_{11} - {\underline{μ}}_{1}}{{\underline{σ}}_{1}} & … & \frac{{x}_{1 k} - {\underline{μ}}_{K}}{{\underline{σ}}_{K}} \\ ⋮ & ⋱ & … \\ \frac{{x}_{N 1} - {μ}_{1}}{{\underline{σ}}_{1}} & … & \frac{{x}_{NK} - {\underline{μ}}_{K}}{{\underline{σ}}_{K}}\end{matrix} ] \qquad ( Equation 6 )$$

where μₖ & σₖ are the mean and standard deviations of the k^{th} spectral measurement of the N samples.

2) Compute the covariance matrix of X (COV(X)=X^{T}X)∈ R^{K×K}, the loading vectors selected pⱼ ∈R^{K×1} (j=1, 2, . . . , the number of the selected PCs) are the eigen vectors of the calculated matrix with the largest eigen values.

3) Calculate the scoring PC vectors (new variables) zⱼ ∈ R^{N×1} by regressing X onto pⱼ and the variables generated from the loading vectors with the highest eigen vectors are only used.

Next, the measured samples represented in terms of the new variables are applied to a supervised multivariate logistic regression model. The model is based on classifying two classes (+ve and −ye) of data based on a training data set. The logistic regression model is based on sigmoid hypothesis function 0<h_{θ}(x)<1, which estimates probability that a given sample with variables x belongs to Class1:

$${h}_{θ} ( x ) = \frac{1}{1 + {e}^{- {θ}^{T} x}} \qquad ( Equation 7 )$$

The model parameter vector θ is calculated by minimizing a logistic regression cost function cost(h_{θ}(x), y) which is trained using a portion of the data set (training set):

$$cost ( {h}_{θ} ( x ) , y ) = \{ \begin{matrix}- log ( {h}_{θ} ( x ) ) & if class 1 ( y = 1 ) \\ - log ( 1 - {h}_{θ} ( x ) ) & if class 0 ( y = 0 )\end{matrix} \qquad ( Equation 8 )$$

The training and the performance check of the model can be performed by assigning 70% of the samples as a training set and the remaining 30% for cross validation. The training set is prepared by selecting 70% of the measured samples randomly (to assure that the set contains samples from both the negative control and viral samples) and then applied to the model to calculate the model parameters. Next, the remaining 30% of the samples (validation set) are applied to the model after calibrating its parameters. Finally, the accuracy of the classification results of the model regarding the validation set is measured by calculating the ratio between the number of correct classified samples to the total number of samples applied during the cross-validation phase only. The training and cross validation process is repeated multiple times, where each time different training and cross validation sets are selected randomly to assure the effectiveness of the results. Then, the average accuracy over the multiple iterations is calculated. In addition, the percentages of True Positive (TP), True Negative (TN), False Positive (FP), and False Negative (FN) are averaged over multiple iterations to ensure that the data set is not skewed, where TP & TN indicate the events of correct viral and correct negative control detection respectively, and FP & FN indicate the events of false viral and negative control detection respectively. In addition, the performance of the chemometrics model may be enhanced through increasing the measured data set, which would facilitate applying more complicated techniques that requires larger data sets than the currently used ones such as Conventional Neural Network (CNN) algorithms.

Neural network models are more sophisticated AI algorithms, and may be used in cases having a large number of input features (variables) where better classification performance is expected to be achieved in case of feeding the network with large data set with multiple input features. The neural network system may include multiple layers. For example, a first layer (input layer) may contain the input features (e.g., variables x₀, x₁, x₂, x₃) and a final layer (output layer) may contain the values of the calculated hypothesis function h_{θ}(x). The layers between the first and final layer are called the hidden layers with each layer including a number of neurons, and the number of hidden layers may change from one model to another. For example, a second layer (Layer 2) may contain neurons α₀⁽²⁾,α₁⁽²⁾,α₂⁽²⁾,α₃⁽²⁾.

The target of the neural network is to calculate the coefficients of the sigmoid function h_{θ}(x) that is used in the classification. In order to calculate h_{θ}(x), the activation values αᵢ^{(j)} (where j is the layer order and i is the neuron order) are calculated for each neuron as a function of the values of the previous layers, and finally h_{θ}(x) is calculated as a function of the activation values of the previous layer. Calculation of the activation values in layer j+1 using the previous layer j may be performed using the matrix of weights Θ^{(j)} using the sigmoid function

$$g ( {θ}^{T} x ) = \frac{1}{1 + {e}^{- {θ}^{T} x}}$$

defined as follows:

a₁⁽²⁾=g(Θ₁₀⁽¹⁾x₀+Θ₁₁⁽¹⁾x₁+Θ₁₂⁽¹⁾x₂+Θ₁₃⁽¹⁾x₃) (equation 9)

a₂⁽²⁾=g(Θ₂₀⁽¹⁾x₀+Θ₂₁⁽¹⁾x₁+Θ₂₂⁽¹⁾x₂+Θ₂₃⁽¹⁾x₃) (equation 10)

a₂⁽²⁾=g(Θ₃₀⁽¹⁾x₀+Θ₃₁⁽¹⁾x₁+Θ₃₂⁽¹⁾x₂+Θ₃₃⁽¹⁾x₃) (equation 11)

Accordingly, the hypothesis function h_{θ}(x) in this example is calculated using these activation values as follows:

h_{θ}(x)=g(Θ₁₀⁽²⁾a₀⁽²⁾+Θ₁₁⁽²⁾a₁⁽²⁾+Θ₁₂⁽²⁾a₂⁽²⁾+Θ₁₃⁽²⁾a₃⁽²⁾ (Equation 12)

It should be noted that the algorithm used to calculate the hypothesis function is denoted as the forward propagation matrix and it can be used for calculation of the hypothesis in the same manner regardless of the number of hidden layers used in the network. The activation values may be written in the vector form as follows:

$${a}^{( 2 )} = g ( {Θ}^{( 1 )} {a}^{( 1 )} ) \qquad ( Equation 13 )$$

$${a}^{( 1 )} = [ {x}_{0} {x}_{1} {x}_{2} {x}_{3} ] \qquad ( Equation 14 )$$

$${Θ}^{( 1 )} = [ \begin{matrix}{Θ}_{10}^{( 1 )} & {Θ}_{11}^{( 1 )} & {Θ}_{12}^{( 1 )} & {Θ}_{13}^{( 1 )} \\ {Θ}_{20}^{( 1 )} & {Θ}_{21}^{( 1 )} & {Θ}_{22}^{( 1 )} & {Θ}_{23}^{( 1 )} \\ {Θ}_{30}^{( 1 )} & {Θ}_{31}^{( 1 )} & {Θ}_{32}^{( 1 )} & {Θ}_{33}^{( 1 )}\end{matrix} ] \qquad ( Equation 15 )$$

The optimum matrix of weights Θ^{j} is calculated by using the training data to minimize the cost function of the neural network J(Θ), where m is the number of training samples, L is the number of layers, and si is the number of neurons in layer 1.

$$J ( Θ ) = \frac{- 1}{m} {\sum }_{i = 1}^{m} {y}^{( i )} log ( {h}_{θ} ( {x}^{i} ) ) + ( 1 - {y}^{( i )} ) log ( 1 - {h}_{θ} ( {x}^{i} ) ) + \frac{λ}{2 m} {\sum }_{l = 1}^{L - 1} {\sum }_{i = 1}^{{s}_{l}} {\sum }_{j = 1}^{{s}_{l} + 1} {( {Θ}_{ij}^{( l )} )}^{2} \qquad ( Equation 16 )$$

The gradient descent iterative algorithm is used to minimize this cost function with initial J(Θ) and

$$\frac{\partial }{\partial {θ}_{ij}^{( l )}} J ( Θ )$$

as its input. To calculate

$$\frac{\partial }{\partial {θ}_{ij}^{( l )}} J ( Θ ) ,$$

a backpropagation algorithm is used. This algorithm is based on calculating the activation values using the forward propagation algorithm with initial value of Θ. Next, the error at the final node δ^{L}=a^{L}−y is calculated, and then the errors in the previous layers are also calculated in a backward manner δ^{l-1}=(Θ^{(t-1)})^{T}δ¹·a^{l-1}·*(1−−a^{l-1}). Finally,

$$\frac{\partial }{\partial {θ}_{ij}^{( l )}} J ( Θ )$$

is calculated using the error calculated iteratively using the training data set as follows:

$$\frac{\partial }{\partial {Θ}_{ij}^{( l )}} J ( Θ ) = \frac{1}{m} {Δ}_{ij}^{( l )} + λ {Θ}_{ij}^{( l )} \qquad ( Equation 17 )$$

where, Δᵢⱼ^{(l)}=Δᵢⱼ^{(l)}+ₐⱼ^{(l)}δᵢ^{(t+1)}, 1 is the order of the layer, j is the order of the neuron in the layer, and I is the order of the data set.

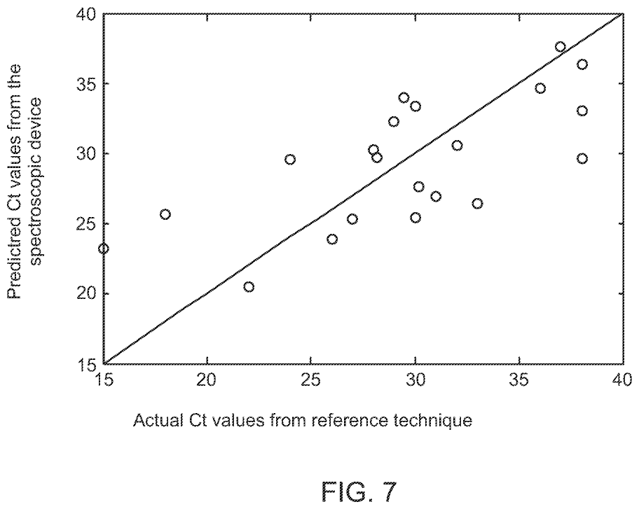

In some examples, the quantification model (calibration model) can be built to predict the viral load or the corresponding cycle threshold (Ct) value. More accurate reference values can be obtained using cell culturing, instead of PCR, at the expense of higher cost and longer time to provide the reference values. An example of results is shown in FIG. 7. Alternative reference methods, such as rapid tests can be used but are less preferred due to lower accuracy. The accuracy of the model is improved by training with a larger number of samples from subjects representing the different clinical states of the subjects.

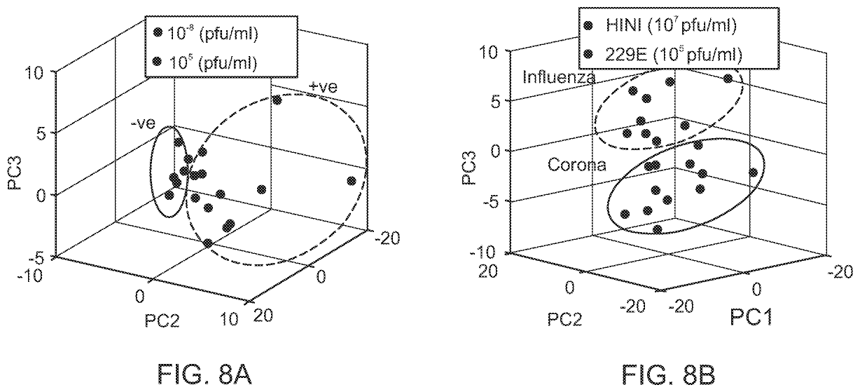

In other examples, the model can be built to cluster/classify the samples whether positive or negative with respect to a certain infection, as shown in FIGS. 8A and 8B. The model shown in FIGS. 8A and 8B extracts the number of features and reduces the dimensionality of the problem to its latent variables. Then, a decision-making mechanism is applied on the clustering. More detailed classification of the positive cases into high, medium, and low viral load is also possible versus the absence of the virus (negative). This can be a regression model versus the Ct values in which high Ct values correspond to low viral load while low Ct values correspond to high viral load. The negative result is accompanied by a no Ct value from the reference technique. Moreover, by training on subjects with different types of infections, such as Corona or Influenza respiratory system infection, the model can classify the infection into different types.

The optical element(s) of the spectroscopic analyzer device shown in FIG. 3 can take various forms. In some examples, the optical element(s) may be configured as a multi-pass cell using high reflectivity mirrors to improve the detection of a sample. For example, multi-pass of light for transmission measurements of a breath sample either trapped on a filter or samples in a compartment can be used. In addition, multi-pass of light may be used for mechanically concentrated analytes on a filter. The location of the sample in the multi-pass cell may be selected to minimize the sample volume. In addition, placing the sample close to a metallic surface may assist in drying the sample by using the metallic surface as a heater. The metallic surface can be part of a mirror (reflector) or a separate part. The mirrors of the multi-pass cell may have different designs, such as spherical, elliptical, or circular section cylinder mirrors. The reflecting surfaces of the multi-pass cell may also take the form of opposed parabolic or parallel pairs, or may have a combination of flat, cylindrical, circular, spiral, or right circular cylinder arrangement of the reflecting surfaces. Different configurations of multi-passing can be used such as circular cells, White cells, heriot cells, and other suitable multi-pass cell configurations.

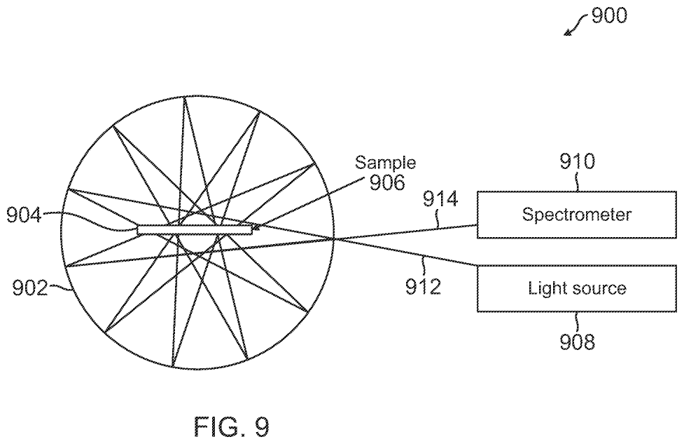

FIG. 9 is a diagram illustrating an example of a spectroscopic analyzer device 900 including a multi-pass cell 902 according to some aspects. The multi-pass cell 902 shown in FIG. 9 is a circular cell having a metallic internal surface forming a reflecting element. A disposable optical component 904 (e.g., a transparent optical window), such as a cover slip, filter, or other suitable transparent optical window having a sample (not shown) thereon may be inserted into the multi-pass cell 902. The transparent optical window 904 may be positioned in a light path within the multi-pass cell 902 having a minimized light spot size. The spectroscopic analyzer device 900 further includes a light source 908 configured to produce incident light (input light) 912 and to direct the input light 912 into the circular multi-pass cell 902. In some examples, the spectroscopic analyzer device 900 may further include one or more input optical coupling elements (e.g., lenses, mirrors, etc.) to couple the input light 912 into the multi-pass cell.

The multi-pass cell 902 is configured to produce output light 914 based on multiple reflections of the input light interacting with the sample on the disposable optical component 904 within the multi-pass cell 902. The spectroscopic analyzer device 900 further includes a spectrometer (and detector) 910 configured to receive the output light from the multi-pass cell 902 and to detect a spectrum of the sample based on the output light 914. In some examples, the spectroscopic analyzer device 900 may further include one or more output optical coupling elements (e.g., lenses, mirrors, etc.) to couple the output light 914 into the spectrometer 910.

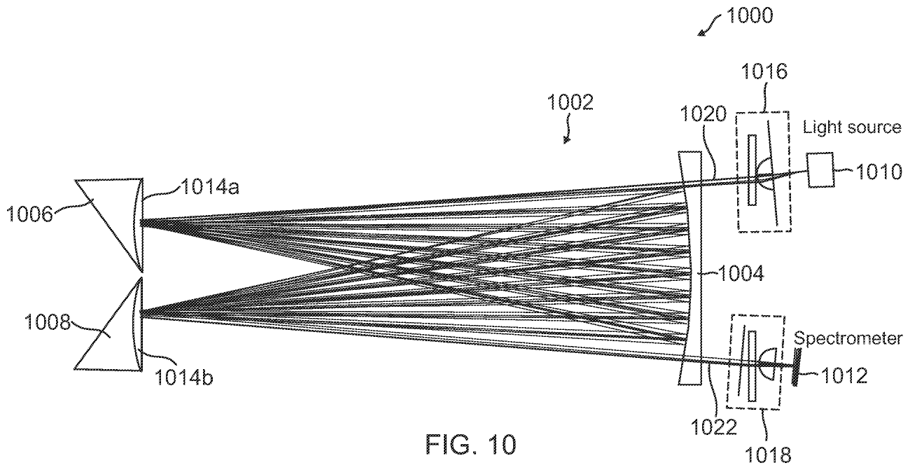

FIG. 10 is a diagram illustrating another example of a spectroscopic analyzer device 1000 including a multi-pass cell 1002 according to some aspects. The multi-pass cell 1002 shown in FIG. 10 is a White cell including a set of three reflecting elements (e.g., mirrors) 1004, 1006, and 1008. White cells reimage the source after each double pass of light up and down the cell, which confines the energy in the cell until the light exits. Due to the confinement, the only source of loss is the non-ideal reflectivity of the mirrors.

Each of the mirrors 1004, 1006, and 1008 may be a spherical mirror. Each of the spherical mirrors 1004, 1006, and 1008 may have the same radius of curvature that is equal to the separation (distance) between a larger spherical mirror 1004 on one side of the cell 1002 and two smaller spherical mirrors 1006 and 1008 on the other side of the cell 1002. For example, spherical mirror 1004 may have a length that is greater than the respective lengths of either of spherical mirrors 1006 and 1008 (e.g., the longer spherical mirror 1004 may have a length that is slightly less than twice the length of either of the shorter mirrors 1006 and 1008). In addition, spherical mirrors 1006 and 1008 may be tilted with respect to one another to provide a small angle between the mirrors 1006 and 1008 selected to maintain the light within the multi-pass cell 1002.

In some examples, at least one of the shorter mirrors (e.g., mirror 1006 and/or 1008) may be removable to form a disposable optical component on which a sample may be placed. In other examples, one or more transparent optical windows 1014a and/or 1014b (e.g., cover slips, filters, or other suitable transparent optical windows), each containing a sample (not shown) may be inserted into the multi-pass cell 1002 and positioned in a light path within the multi-pass cell 1002 having a minimized light spot size. For example, the transparent optical window(s) 1014a and/or 1014b may be placed adjacent to the shorter mirrors 1006 and/or 1008 in the light path. The spectroscopic analyzer device 1000 further includes a light source 1010 configured to produce incident light (input light) 1020 and to direct the input light 1020 into the multi-pass cell 1002. In some examples, as shown in FIG. 10, the spectroscopic analyzer device 1000 may further include one or more input optical coupling elements 1016 (e.g., lenses, mirrors, etc.) to couple the input light 1020 into the multi-pass cell 1002.

Multiple reflections of the input light 1020 between the longer spherical mirror 1004 and each of the shorter spherical mirrors 1006 and 1008 may then occur making at least two passes up and down the multi-pass cell 1002. The multi-pass cell 1002 is thus configured to produce output light 1022 based on the multiple reflections of the input light interacting with the sample on the disposable optical component (e.g., one or more of the shorter mirrors 1006 and/or 1008 or one or more transparent optical windows 1014a and/or 1014b) within the multi-pass cell 1002. The spectroscopic analyzer device 1000 further includes a spectrometer (and detector) 1012 configured to receive the output light 1022 from the multi-pass cell 1002 and to detect a spectrum of the sample based on the output light 1022. In some examples, the spectroscopic analyzer device 1000 may further include one or more output optical coupling elements 1018 (e.g., lenses, mirrors, etc.) to couple the output light 1022 into the spectrometer 1012.

In some examples, one or more of the optical element(s) shown in FIG. 3 may be configured as an attenuated total internal reflection (ATR) element. ATR is a spectroscopic technique that can be used in Fourier transform infrared (FTIR) spectrometers that provides wide spectral range of operation. ATR is a type of reflection coupling which depends on internal reflection of light inside a high refractive index material while placing a sample in intimate contact with the material. ATR has been used as an effective way to analyze fluid and solid samples based on the phenomenon of total internal reflection of light at the boundary between two media. Typically, a high refractive index material such as zinc selenide (ZnSe), germanium (Ge), diamond, or silicon, referred to as an ATR crystal, ATR internal reflection element (IRE), or ATR element, is illuminated with an IR source, while the sample covers the ATR crystal and is in intimate contact therewith. If the angle of input light is higher than a critical angle at the boundary between the ATR crystal and the sample, light is totally internally reflected and a special type of electromagnetic wave, called an evanescent wave, is formed on the sample side. The sample absorbs some of the intensity of the evanescent wave due to molecular vibrations at certain wavelengths. Hence, the intensity of reflected light is attenuated relative to the incoming intensity. The output light of the crystal may then be coupled to a Fourier Transform Infrared (FTIR) system, as an example for spectroscopic techniques, which may be based on a Michelson interferometer, and then to a broadband IR detector to analyze the sample spectrum.

ATR enables liquid analysis with short and consistent effective interaction length with samples. Hence, it prevents the strong solvent absorption features from attenuating the transmitted IR intensity and provides high reproducibility of measurements. This is in contrast to transmission mode configurations, where it can be difficult to ensure the reproducibility of thin spacers' thickness and prevention of air bubbling while filling the transmission cell. ATR further allows minimal sample preparation for liquids and solids as the samples are placed in direct contact with a crystal of high refractive index materials. In addition, non-invasive sample measurements (e.g., of skin) can be also carried out by direct contact between skin and an ATR element. For example, different biomarkers can be detected from the outer layers of the skin such as inflammatory protein-like biomarkers, e.g. Cytokines, or lipid biomarkers responsible for the natural defense barrier of the epidermis, including cholesterol, ceramides, fatty acids. The effect of cosmetics products and the detection of collagen is also possible.

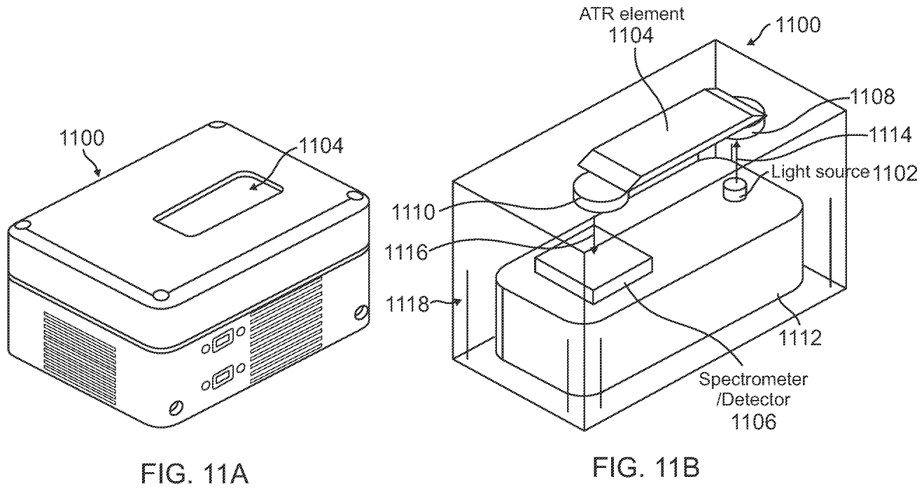

FIGS. 11A and 11B illustrate an example of a spectroscopic analyzer device 1100 including an ATR element 1104 according to some aspects. In some examples, the ATR element 1104 may be a disposable optical component on which a sample (not shown) may be placed. The ATR element 1104 may be a ZnSe, Ge, diamond, silicon, or other ATR crystal. In some examples, the ATR element material may be selected based on one or more properties of the biological sample (e.g., the sample pH level).

The spectroscopic analyzer device 1100 further includes a light source 1102, a spectrometer/detector 1106, optical coupling elements 1108 and 1110, an electronic board 1112 including, for example, power and control circuitry, and an enclosure 1118 surrounding the light source 1102, ATR element 1104, spectrometer/detector 1106, optical coupling element 1108 and 1110, and electronic board 1112. In some examples, the light source 1102 may be a tungsten-IR light source. The wavelength range may be selected based on the infrared spectral features of the biological sample under test. The optical coupling elements 1108 and 1110 (e.g., lenses or mirrors) can be used to improve light coupling all along the path from the light source 1102 to the spectrometer/detector 1106.

In some examples, the detector 1106 may be a photo detector, such as Pbs, PbSe, MCT, thermal or any other device for converting the optical power to an electrical signal. In some examples, the spectrometer 1106 may be a MEMS-based Michelson interferometer, and the displacement range of the moving micromirror is chosen to resolve the spectral features in the spectrum of the biological sample. For example, 180 μm corresponding to a spectral resolution of 33 cm⁻¹, while reducing the distance to one half leads to a resolution of 66 cm⁻¹ The spectrometer/detector 1106 can be assembled in a single package for a more reliable alignment and less noise signal coupling on the detector. In addition, the light source 1102 and package containing the spectrometer and detector 1106 may be assembled on the same electronic board (e.g., printed circuit board (PCB)) 1112.

In some examples, the enclosure 1118 of the spectroscopic analyzer device 1100 can be a good thermal dissipater (heat sink) made of metallic or high thermal conductive polymer-based material to manage the thermal stability of the device. One or more fans (not shown) can also be included to circulate the air.

In an example operation, the light source 1102 is configured to produce incident light 1114 (input light) and to direct the input light 1114 to the ATR element 1104 via the input optical coupling element(s) 1108. The ATR element 1104 may be a single-reflection ATR element or a multiple-reflections ATR element. The ATR element 1104 is designed to produce total internal reflection of the input light 1114 at the interface between the ATR element 1104 and the sample (not shown). For example, the ATR element 1104 may have a size (dimensions, thickness, etc.) and shape (e.g., V-shaped or angled input and output interfaces) configured to produce an angle of the input light 1114 that is higher than a critical angle at the interface between the ATR element 1104 and the sample. The resulting evanescent wave produced in the sample based on the total internal reflection of the input light 1114 attenuates the input light 1114 to produce output light 1116 that may be input to the spectrometer/detector 1106 via the output optical coupling element(s) 1110.

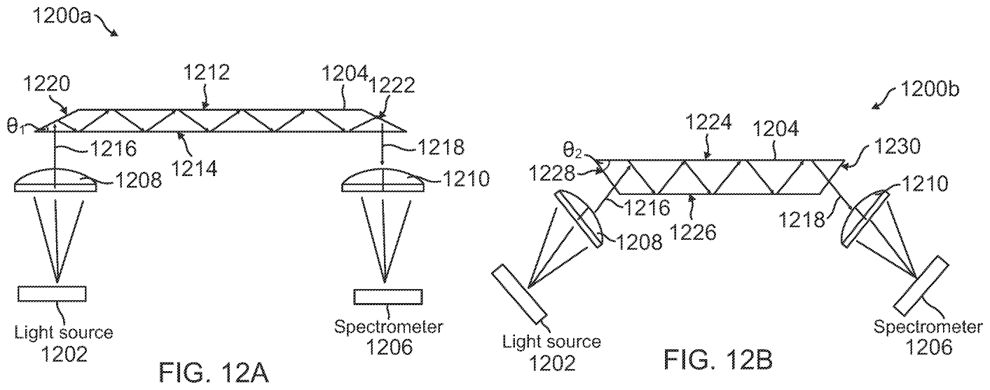

FIGS. 12A and 12B are diagrams illustrating other examples of spectroscopic analyzer devices 1200a and 1200b including an ATR element 1204 according to some aspects. The spectroscopic analyzer device 1200a or 1200b further includes a light source 1202, a spectrometer 1206 (e.g., a spectrometer and detector), and optical coupling elements (e.g., lenses) 1208 and 1210. The light source 1202 is configured to produce incident light 1216 that is directed into the ATR element 1204 via the input optical coupling element 1208. The ATR element 1204 is designed to produce total internal reflection of the input light 1216 at an interface between the ATR element 1204 and a sample (not shown) on the ATR element 1204. The resulting evanescent wave produced in the sample based on the total internal reflection of the input light 1216 attenuates the input light 1216 to produce output light 1218 that may be input to the spectrometer/detector 1206 via the output optical coupling element 1210.

In the examples shown in FIGS. 12A and 12B, the ATR element 1204 is a multiple-reflections ATR element. In the example shown in FIG. 12A, the ATR element (e.g., ATR crystal) 1204 includes a first (top) surface 1212 (e.g., top ATR crystal face) forming an interface with the sample and a second (bottom) surface 1214 (e.g., bottom ATR crystal face) opposite the first surface. In addition, the ATR element 1204 includes opposing angled side surfaces 1220 and 1222 (e.g., side ATR crystal faces). Each of the opposing angled side surfaces 1220 and 1222 has a face angle θ₁ with respect to the bottom surface 1214 greater than the critical angle for total internal reflection. Thus, the opposing angled side surfaces 1220 and 1222 are configured to produce total internal reflection of the input light 1216 within the ATR element 1204 and to output of the output light 1218 via the bottom surface 1214. For example, the input light 1216 is directed into the ATR element 1204 through the bottom surface 1214 towards the entrance angled side surface 1220, where the input light is totally internally reflected back towards the bottom surface 1214 at an angle with respect to the bottom surface 1214 greater than the critical angle for total internal reflection. The input light 1216 then undergoes multiple total internal reflections between the top surface 1212 and the bottom surface 1214 until the light reaches the exit angled side surface 1222, which totally internally reflects the output light 1218 normal to the bottom surface 1214 for output from the ATR element 1204 to the spectrometer 1206 via the output coupling element 1210.

In the example shown in FIG. 12B, the ATR element 1204 also includes top and bottom surfaces 1224 and 1226, along with angled side surfaces 1228 and 1230. In the configuration shown in FIG. 12B, the input light 1216 is directed into the ATR element 1204 via the angled side surface 1228. The angled side surfaces 1228 and 1230 have a face angle θ₂ with respect to the top surface 1224 configured to produce total internal reflection of the input light 1216 within the ATR element 1204 and to output the output light 1218 via the exit angled side surface 1230. For example, the input light 1216 is directed into the ATR element 1204 through the entrance angled side surface 1228 towards the top surface 1228, where the input light is totally internally reflected back towards the bottom surface 1226 at an angle with respect to the top surface 1224 greater than the critical angle for total internal reflection. The input light 1216 then undergoes multiple total internal reflections between the top surface 1224 and the bottom surface 1228 until the light reaches the exit angled side surface 1230, where the output light 1218 is output from the ATR element 1204 to the spectrometer 1206 via the output coupling element 1210.

The effective interaction length with the sample may be controlled by controlling both the length of the ATR element (e.g., ATR crystal), which affects the number of reflections, and the face angle (θ₁ or θ₂) of the ATR crystal, which affects the angle of incidence on the crystal-sample interface. In some examples, the ATR crystal face surfaces can be coated with a reflective coating to trap the light in the ATR crystal and amplify the optical path length of interaction with the sample. In some examples, the light may be reflected back at the exit face (e.g., exit face surface 1230) of the crystal and travel multiple times through the ATR crystal 1204 before eventually being output at the exit face 1230 or the entrance face 1228 and collected at the spectrometer 1206. Hence, the effective number of reflections may be more than the calculated number from the crystal's geometry of the direct path and the given angle of incidence.

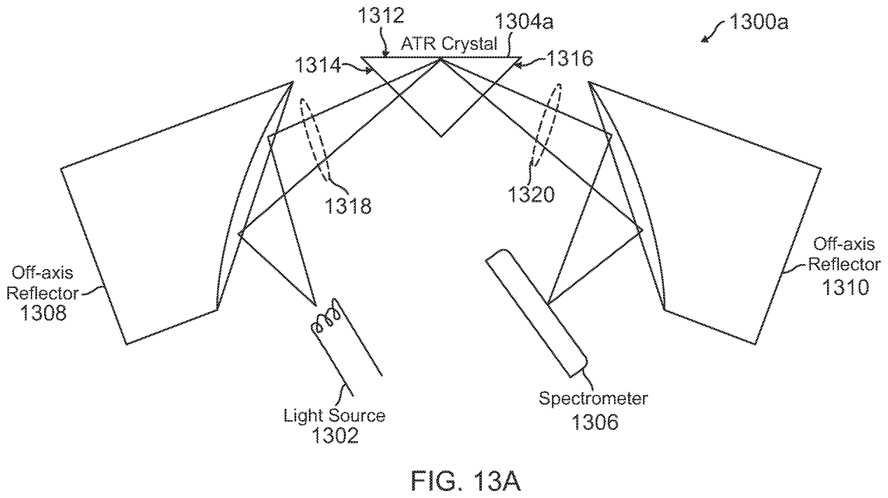

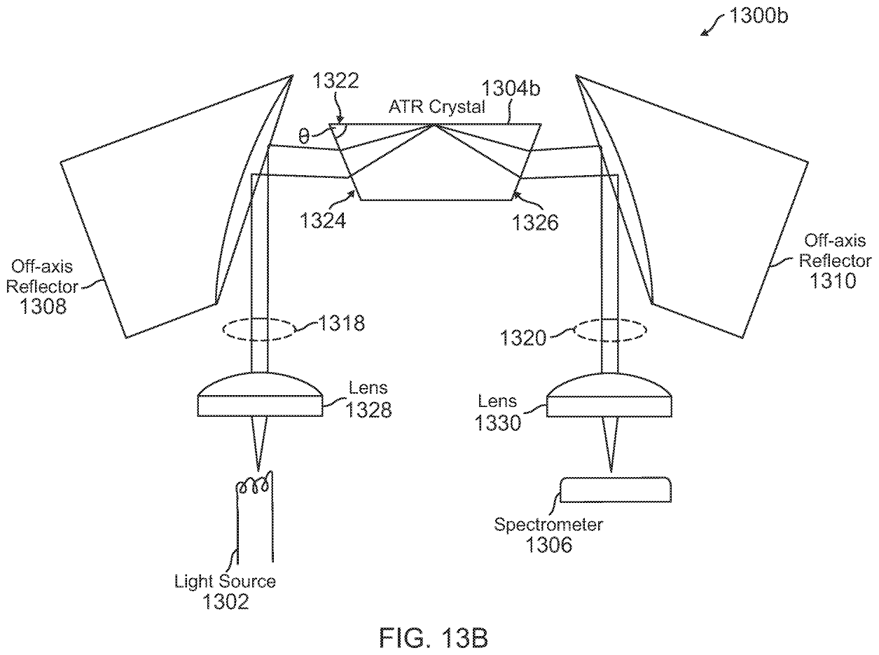

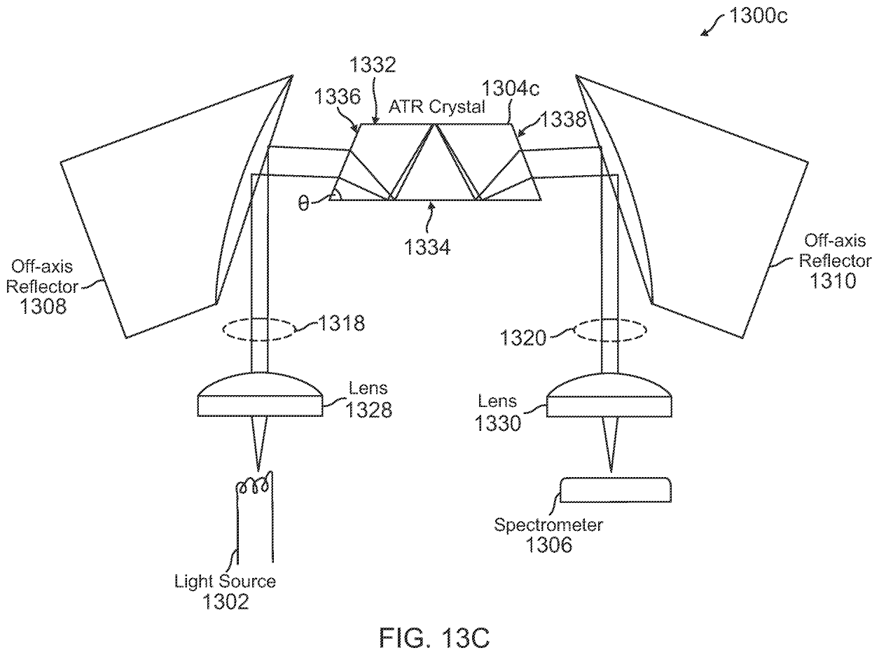

FIGS. 13A, 13B, and 13C are diagrams illustrating other examples of spectroscopic analyzer devices 1300a, 1300b, and 1300c including an ATR element 1304 according to some aspects. The spectroscopic analyzer device 1300a, 1300b, or 1300c further includes a light source 1302, a spectrometer 1306 (e.g., a spectrometer and detector), and optical coupling elements 1308 and 1310. The optical coupling elements 1308 and 1310 may be off-axis reflectors, such as off-axis parabolic mirrors.

For some biological applications, the sample volume should be minimized down to a few micro liters or nano liters. In this case, single reflection with a very small optical spot size is needed. As such, in the examples shown in FIGS. 13A, 13B, and 13C, the ATR element 1304 is a single reflection ATR element.

For example, as shown in FIG. 13A, the light source 1302 is rotated by 45°, such that incident light 1318 (e.g., input light) produced by the light source 1302 is illuminating in all directions. The input light 1318 can be collected using, for example, a 90° off-axis reflector 1308 rotated as well by 45°, so that the focused input light 1318 enters the ATR element 1304a (ATR crystal) with a 45° incidence angle. The ATR crystal 1304a has a prism shape with a top surface 1312 in contact with a sample (not shown) and two side surfaces 1314 and 1316, each having a respective 45° face angle. The input light 1318 from the off-axis reflector 1308 enters the ATR element 1304a through the entrance side surface 1314, where the light 1318 is directed towards the top surface 1312. In some examples, the ATR crystal 1304a has a 45° face angle to totally internally reflect the input light 1318 from the top surface 1312 of the ATR crystal with a 45° incidence angle to produce high penetration depth inside the sample. The resulting output light 1320 is directed outside the ATR crystal 1304a from the exit side surface 1316 towards the off-axis reflector 1310. In some examples, the off-axis reflector 1310 is a 90° off-axis reflector positioned with 45° tilt to redirect the output light 1320 downwards with a 45° angle, where the output light 1320 can be collected by a rotated spectrometer 1306.

The two off-axis reflectors 1308 and 1310 may be identical or each reflector 1308 and 1310 may be optimized for performance. For example, the reflectors 1308 and 1310 may be optimized to collect as much power from the light source and focus the highest power into the spectrometer 1306, limited by its throughput. In some examples, the light source 1302 can be mounted with a vertical axis and the input light 1318 may be collected using a 90° off-axis reflector 1308 that is tilted by 22.5° to focus the light 1318 on the ATR crystal 1304a with a 45° incidence angle. The input light 1318 incident to the ATR crystal 1304a and interacting with the sample can focused, defocused, or collimated by changing the distance between the light source 1302 and the off-axis reflector 1308 to control the optical spot size at the sample.

In the example shown in FIG. 13B, the input light radiated by the light source 1302 is collimated using a lens 1328. The collimated light 1318 is focused using a 90° off-axis reflector 1308 to the ATR crystal 1304b. The ATR crystal 1304b has a trapezoidal shape with θ(>90° as a face angle. In addition, the ATR crystal 1304b includes a top surface 1322 in contact with the sample and two opposing angled side surfaces 1324 and 1326. The light 1318 is input to the ATR crystal through the entrance side surface 1324, where the light is redirected based on the face angle and then totally reflected from the top surface 1322 of the ATR crystal 1304b. The total internal reflected light is directed outside the ATR crystal 1304b from the exit side surface 1326 as output light 1320. The output light 1320 is directed to another 90° off-axis reflector 1310 positioned to collimate the output light 1320 downwards and then focus the output light 1320 to the spectrometer 1306 using a second lens 1330.

In the example shown in FIG. 13C, the input light 1318 from the light source 1302 is again collimated using the lens 1328 and the resulting collimated input light 1318 is focused using the 90° off-axis reflector 1308 to the ATR crystal 1304c. The ATR crystal 1304c shown in FIG. 13C also has a trapezoidal shape with θ(>90° as a face angle. In addition, the ATR crystal 1304c includes a top surface 1332 in contact with the sample, a bottom surface 1334, and two opposing angled side surfaces 1336 and 1338. The light 1318 is input to the ATR crystal 1304c through the entrance side surface 1336, where the light 1318 is redirected based on the face angle from the bottom surface 1334, and then totally internally reflected from the top surface 1332 of the ATR crystal 1304c. The total internal reflected light is then reflected off the bottom surface 1334 and directed outside the ATR crystal 1304c from the exit side surface 1338 as output light 1320. The output light 1320 is directed to the other 90° off-axis reflector 1310 positioned to collimate the output light 1320 downwards and then focus the output light 1320 to the spectrometer 1306 using the second lens 1330. In the examples shown in FIGS. 13B and 13C, the trapezoidal shape of the ATR crystal 1304b and 1304c may result in easy handling and assembly of the ATR element 1304b and 1304c into the spectroscopic analyzer device 1300b and 1300c.

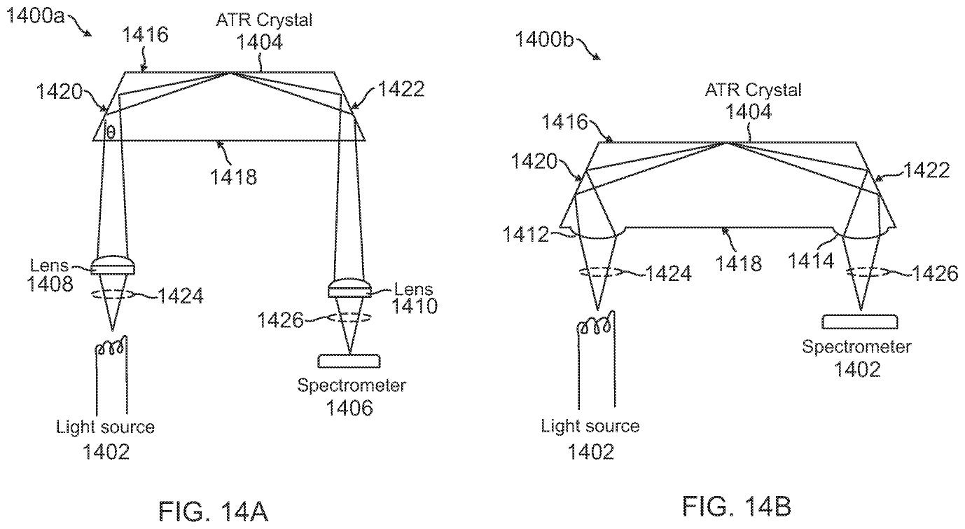

FIGS. 14A and 14B are diagrams illustrating other examples of spectroscopic analyzer devices 1400a and 1400b including an ATR element 1404 according to some aspects. The spectroscopic analyzer device 1400a or 1400b further includes a light source 1402, a spectrometer 1406 (e.g., a spectrometer and detector). In the example shown in FIG. 14A, the spectroscopic analyzer device 1400a may further include optical coupling elements 1408 and 1410. The optical coupling elements 1408 and 1410 may be, for example, lenses. In this example, incident light 1424 (input light) from the light source 1402 is radiated and collected and focused using the lens 1408.

In each of FIGS. 14A and 14B, the ATR element 1404 (ATR crystal) is a single reflecting ATR element that has a trapezoidal shape with θ(>45° as a face angle. The ATR crystal 1404 further includes a top surface 1416 in contact with a sample (not shown), a bottom surface 1418 opposite the top surface 1416, and two angled opposing side surfaces 1420 and 1422. The input light 1424 enters the ATR crystal 1404 through the bottom surface 1418 towards a first side surface 1420, where the light is subjected to a first total internal reflection thereof based on the face angle to direct the light to the top surface 1416. The light is then subjected to a single total internal reflection from the top surface 1416 of the ATR crystal 1404 to produce output light 1426. The output light 1426 is directed outside the ATR crystal from the bottom surface 1418 after being subjected to a second total internal reflection thereof from a second side surface 1422 of the ATR crystal 1404.

In the example shown in FIG. 14A, the resulting output light 1426 can be focused to the spectrometer 1406 using the lens 1410. In some examples, the lenses 1408 and 1410 may be positioned to collimate the light instead of focusing the light. In this example, all of the output light 1426 can be collimated during the propagation inside the ATR crystal 1404. In another configuration, as shown in FIG. 14B, instead of using lenses 1408 and 1410, the light can be collected and focused using respective curved surfaces 1412 and 1414 monolithically integrated in the ATR crystal 1404. The curved surfaces 1412 and 1414 correspond to curved portions of the bottom surface 1418 of the ATR element 1404.

In other configurations, the light source output may first be coupled to the spectrometer and the resulting interference beam may be input to the ATR element. The output light from the ATR element may then be provided to the detector. In this configuration, the detector can be modular and changed by the user to change the wavelength range of operation based on the analyses sample.

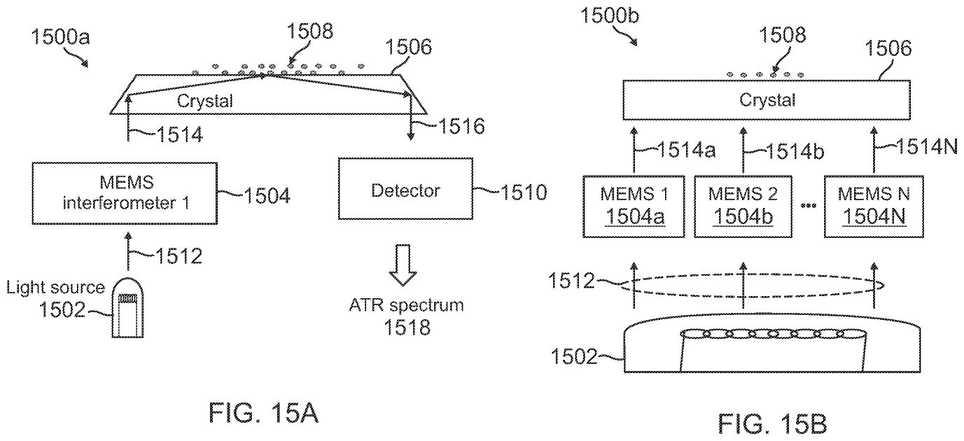

FIGS. 15A and 15B are diagrams illustrating examples of a spectroscopic analyzer device 1500a and 1500b including a spectrometer in the light path between a light source 1502 and an ATR element 1506 according to some examples. In some examples, the spectrometer may be a MEMS interferometer 1504. In the example shown in FIG. 15A, the light path can be based on injecting incident light 1512 from the light source 1502 into the MEMS interferometer 1504 to produce an interference beam 1514 that is input to the ATR element 1506 (ATR crystal) as input light. The ATR element 1506 may be a single reflecting ATR element, as shown in FIG. 15A or a multiple reflecting ATR element. The input light 1514 (interference beam) is totally internally reflected at an interface between the ATR crystal 1506 and a sample 1508 on the ATR crystal 1506 (e.g., on a top surface of the ATR crystal). The resulting output light 1516 is coupled to a detector 1510 configured to produce a spectrum 1518 (ATR spectrum) of the sample. In this example, coupling of the incident light 1512 from the light source 1502 to the interferometer 1504 is not affected by the sample 1508, and is maximized by bringing the interferometer 1504 in close proximity with the light source 1502.

Since a compact miniaturized MEMS interferometer may have a limited optical throughput, as shown in the example of FIG. 15B, multiple MEMS interferometers (e.g., MEMS 1 1504a, MEMS 2 1504b, MEMS N 1504N) can be distributed spatially to capture more power from the light source. For example, the incident light 1512 from the light source 1502 can be coupled into each of the MEMS interferometers 1504a, 1504b, . . . , 1504N, and the resulting interference beams 1514a, 1514b, . . . , 1514N produced by the MEMS interferometers 1504a, 1504b, . . . , 1504N can be input to the ATR crystal 1506. Thus, the interference beams 1514a, 1514b, . . . , 1514N collectively form the input light to the ATR element 1506. The output light resulting from total internal reflection of the interference beams 1514a, 1514b, . . . , 1514N (input light) within the ATR crystal 1506 can be captured by a detector (not shown).

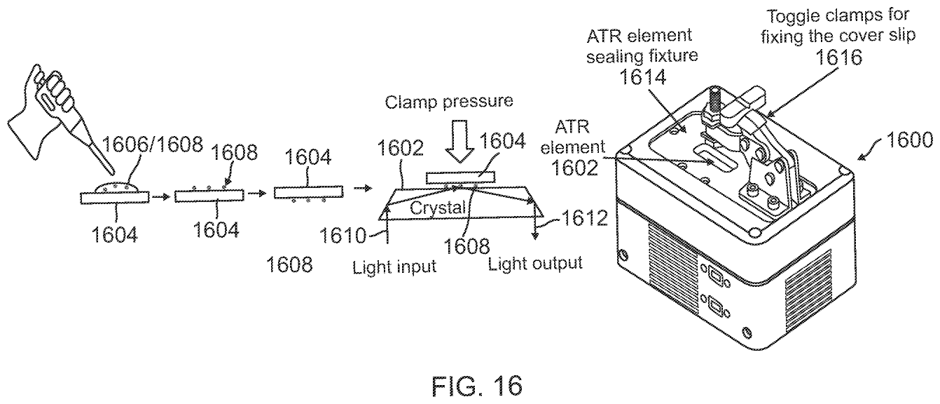

FIG. 16 is a diagram illustrating another example of a spectroscopic analyzer device 1600 including an ATR element 1602 according to some aspects. In the example shown in FIG. 16, the ATR element 1602 (ATR crystal) can be fixed and sealed within the spectroscopic analyzer device 1600 using an ATR element sealing fixture 1614. In this example, the disposal optical component of the optical element(s) shown in FIG. 3 may include a disposable cover slip 1604. A sample 1608 may be placed on the cover slip 1604. In some examples, an array of cover slips can be used to increase the throughput of the analysis. In examples in which the sample 1608 is contained within a media 1606, the sample 1608 can optionally be dried to evaporate the media 1606. The cover slip 1604 may then be inverted (flipped) and applied on the ATR crystal 1602 to position the sample 1608 adjacent to the ATR element 1602. As such, the analyte (sample 1608) is in contact with the ATR crystal 1602 to enable input light 1610 to interact with the sample 1608 to produce output light 1612 that may be measured. One or more clamps 1616 (e.g., toggle clamps) can be used to fix the cover slip 1604 and press the cover slip 1604 in close contact with the ATR crystal 1602 to improve the repeatability of the measurements. In some examples, the cover slip 1604 can be a paper sheet, filter sheet, deformable polymer/plastic material, glass, infrared transparent material, or other suitable material.

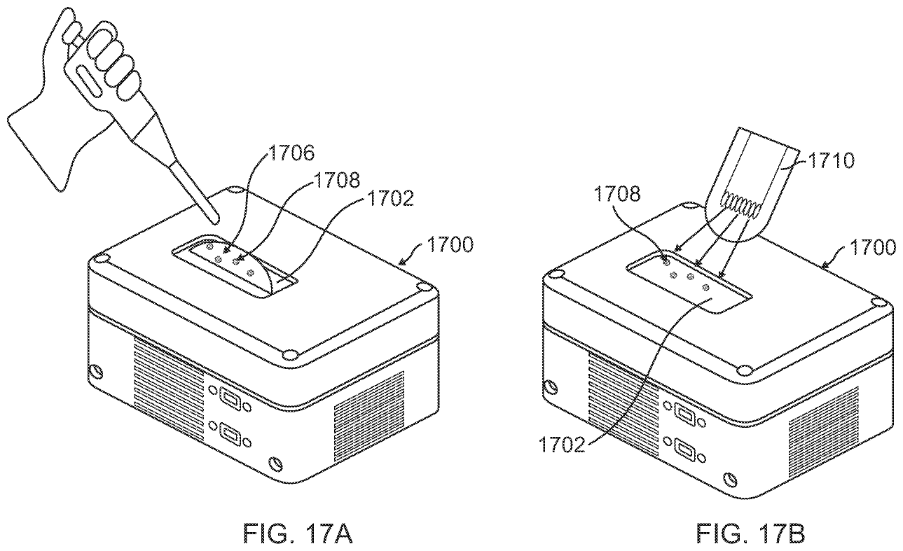

FIGS. 17A and 17B are diagrams illustrating another example of a spectroscopic analyzer device 1700 including an ATR element 1702 according to some aspects. In the example shown in FIG. 17A, a media 1706 containing a sample 1708 can be placed on the top of ATR element 1702 (ATR crystal). As shown in FIG. 17B, the sample 1708 can then be dried directly using heating lamp 1710 or other drying agent at a controlled temperature (e.g., 40° C.) to evaporate the media 1706. In some examples, a spectrum of air can be used as a background before each measurement. For example, for each measurement, several scans can be recorded. In addition, alcohol and distilled water, for instance, can be used for cleaning between successive samples. Measurement of each sample 1708 can be repeated multiple times to train the calibration model of the AI engine. For example, the spectrum can be continuously measured during the drying period. The temperature of the sample can be monitored using a temperature sensor during the drying and the measurements. The captured spectra can all be fed into the AI engine to improve the calibration model accuracy as the sample spectra is measured at different temperatures and water content conditions.

In general, a source of heat and a drying agent can be used to remove the vapor produced by the drying process. For example, instead of a heating lamp 1710, a gas stream, e.g., air, can apply the heat by convection and carry the vapor as humidity. As another example, vacuum drying may be used, in which the heat is supplied by conduction or radiation, while the vapor produced is removed by the vacuum. Instead of drying the sample 1708, mechanical extraction of the solvent by filtration or centrifugation can be used as a draining mechanism.

FIG. 18 is a diagram illustrating another example of a spectroscopic analyzer device including an ATR element 1802 according to some aspects. The spectroscopic analyzer device includes a top cover 1800 having self-alignment grooves 1804 facilitating insertion and removal of a detachable ATR element 1802 (ATR crystal). The detachable ATR crystal 1802 can be removed to be cleaned or for sample application and drying and then attached again easily with no need for re-alignment. This enables performing drying or any other processing in batch mode on multiple samples at the same time, then attaching the multiple ATR crystals 1802 one by one for acquiring the spectral signal. In some examples, the detachable ATR crystal 1802 may be disposable. For example, the ATR crystal 1802 made of silicon or any other material that can be used one time only and then thrown away.

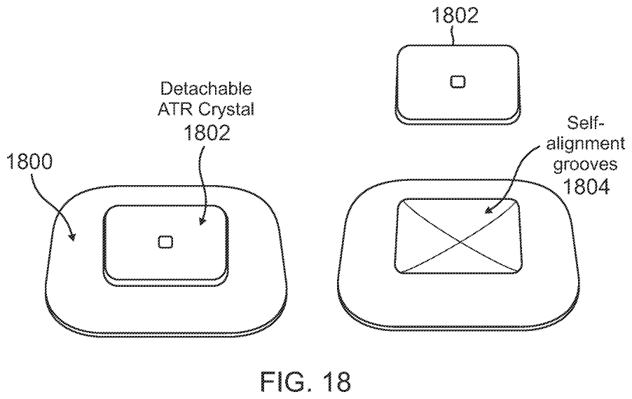

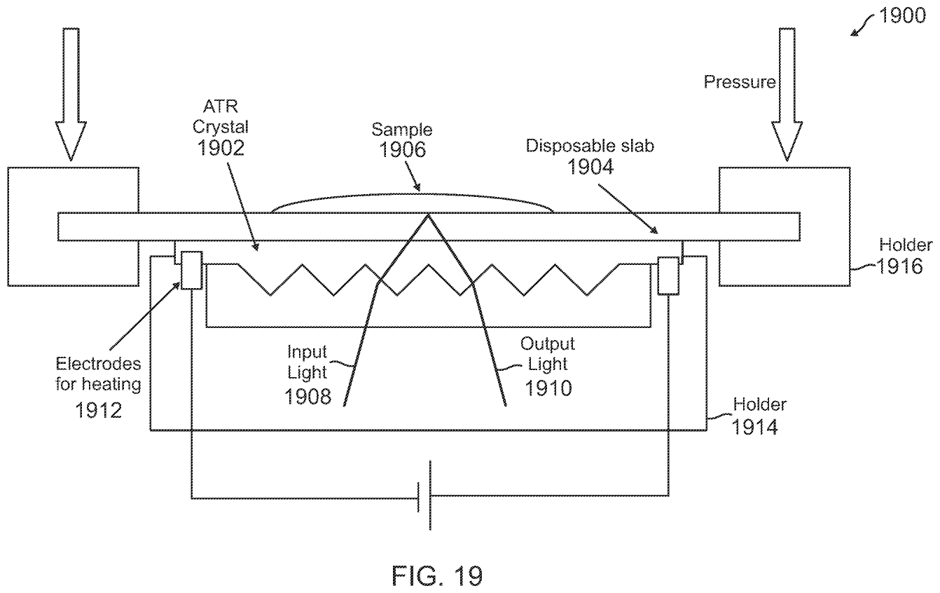

FIG. 19 is a diagram illustrating another example of a spectroscopic analyzer device 1900 including an ATR element 1902 according to some aspects. In the example shown in FIG. 19, the ATR element 1902 (ATR crystal) is fixed. In this example, the spectroscopic analyzer device 1900 includes a disposable and removable slab 1904 of silicon or any other material. Thus, the disposable slab 1904 forms the disposable optical component of the optical element(s) shown in FIG. 3. A sample 1906 may be placed above the slab 1904. Holders 1914 and 1916 may be used to apply pressure to the slab 1904 to ensure the contact between the ATR crystal 1902 and the slab 1904, thus reducing losses and ensuring good transmission to the slab 1904 where input light 1908 will be total internal reflected from the slab interface with the sample 1906 to produce output light 1910 for measurement. For sample drying, heaters (e.g., electrodes 1912) may be connected electrically to the ATR crystal/slab 1902/1904 introducing the heat to the sample 1906.

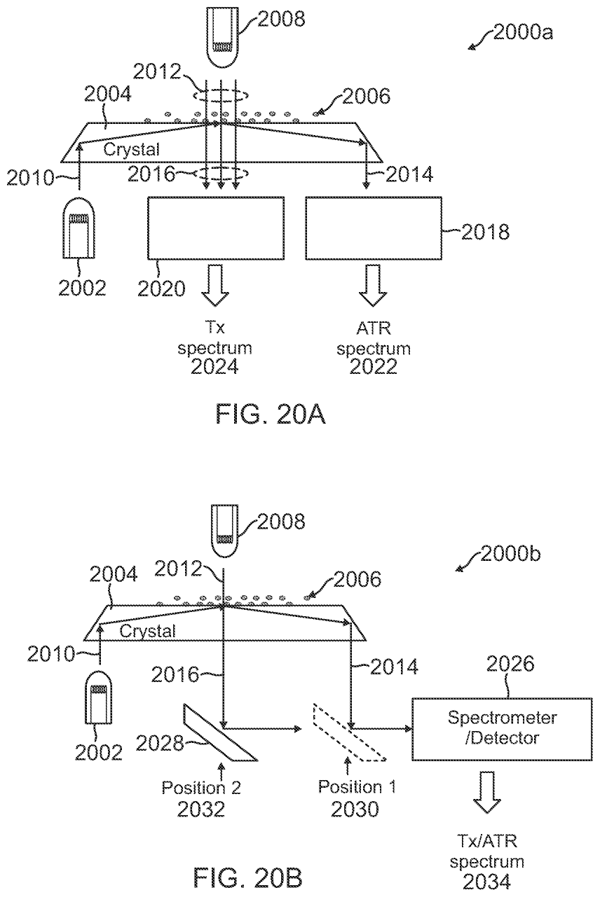

FIGS. 20A and 20B are diagrams illustrating another example of a spectroscopic analyzer device 2000a and 2000b including an ATR element 2004 according to some aspects. In the example shown in FIGS. 20A and 20B, a sample 2006 can be measured in both an ATR mode and a transmission mode simultaneously. The transmission mode may be used to capture an accumulation of the analyte (sample) 2006 in the vertical direction on the top of the ATR element 2004 (ATR crystal). The ATR mode may be used to guarantee a fixed optical path length for the interaction with the sample 2006.

To facilitate simultaneous ATR and transmission modes, the spectroscopic analyzer device 2000 may include two light sources 2002 and 2008. A first light source 2002 may be configured to produce first incident light (input light) 2010 that is input to the ATR element 2004 for total internal reflection thereof on a top surface of the ATR element forming an interface with the sample 2006 (ATR/sample interface) to produce first output light 2014. A second light source 2008 may be configured to produce second (additional) input light 2012 and to direct the second input light through the sample 2006 and the ATR element 2004 to produce second output light 2016.

In the example shown in FIG. 20A, the spectroscopic analyzer device 2000a includes two spectrometers 2018 and 2020 (two spectrometers/detectors), each configured to receive a respective one of the output lights 2014 and 2016. For example, a first spectrometer 2018 may be configured to receive the first output light 2014 in ATR mode, while a second spectrometer 2020 may be configured to receive the second output light 2016 in the transmission mode. The first spectrometer 2018 may be configured to produce a first interference beam, which may be detected by a detector (not shown) to produce an ATR spectrum 2022. The second spectrometer 2020 may be configured to produce a second interference beam, which may be detected by a detector (not shown) to produce a transmission spectrum 2024.

In the example shown in FIG. 20B, a single spectrometer 2026 (spectrometer/detector) may be used. In this example, an optical switching device 2028, such as a moving mirror, is used to switch the output light 2014 or 2016 to the spectrometer 2026. For example, in a first position 2030 of the moving mirror 2028, the moving mirror 2028 may couple the output light 2014 in ATR mode to the spectrometer 2026, while in a second position 2032 of the moving mirror 2028, the moving mirror 2028 may couple the output light 2016 in transmission mode to the spectrometer 2026. The spectrometer 2026 may then produce a combined transmission/ATR spectrum 2034 of the sample. In some examples, the moving mirror 2028 can be integrated into a single chip with the MEMS interferometer (spectrometer 2026) and driven by a MEMS actuator. In other examples, the moving mirror 2028 can be a discrete component driven by a discrete motor.

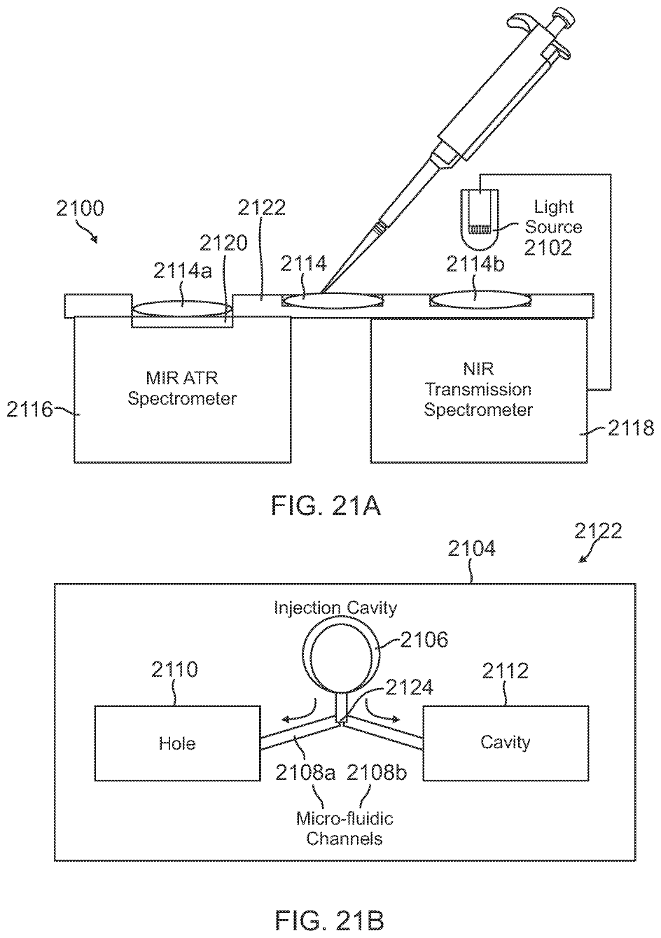

FIGS. 21A and 21B are diagrams illustrating another example of a spectroscopic analyzer device 2100 including an ATR element 2120 according to some aspects. In the example shown in FIGS. 21A and 21B, the spectroscopic analyzer device 2100 includes two spectrometers 2116 and 2118. A first spectrometer 2116 can operate in an ATR mode, while a second spectrometer 2118 can operate in a transmission mode. Both spectrometers 2116 and 2118 can operate simultaneously to measure a same sample droplet 2114. In some examples, one of the spectrometers (e.g., the first spectrometer 2116) may operate in a MIR (mid infrared) range and the other spectrometer (e.g., the second spectrometer 2118) may operate in the NIR (near infrared) range to increase the spectral range and consequently, improve the detection accuracy.

The spectroscopic analyzer device 2100 may further include a sample slide 2122 that employs a fluidic splitter 2104. The sample slide 2122 may correspond to the disposable optical component of the optical element(s) shown in FIG. 3. The fluidic splitter 2104 includes an injection cavity 2106 configured to receive the sample droplet 2114, micro-fluidic channels 2108a and 2108b, an output hole 2110 for ATR measurement in ATR mode, and an output cavity 2112 for transmission measurement in transmission mode. The fluidic splitter 2104 may be configured to split the sample 2114 into a first sample portion 2114a and a second sample portion 2114b using the first micro-fluidic channel 2108a and the second micro-fluidic channel 2108b. For example, the sample 2114 may be injected into the injection cavity 2106. The sample 2114 may then flow to a junction 2124 between the first micro-fluidic channel 2108a and the second micro-fluidic channel 2108b, where the sample 2114 is split such that a respective portion of the sample 2114 is directed into each of the micro-fluidic channels 2108a and 2108b. The fluidic splitter 2104 is further configured to direct the first sample portion 2114a to the output hole 2110 via the first micro-fluidic channel 2108a for sample measurement in ATR mode, and to direct the second sample portion 2114b to the output cavity 2112 via the second micro-fluidic channel 2108b for sample measurement in transmission mode.

In some examples, the spectroscopic analyzer device 2100 may include multiple spectrometers of the same type (e.g., multiple MIR ATR spectrometers and/or multiple NIR transmission spectrometers) to reduce the testing time, achieving the same SNR in 1/\sqrt{N} the scan time, where N is the number of spectrometers of the same type. In some examples, the sample slide may be configured as a micro-fluidic chip. In this example, the micro-fluidic chip may be used to optionally mix the sample 2114 with other chemicals before the analysis or for pre-concentration of the sample 2114 using mechanical filters.

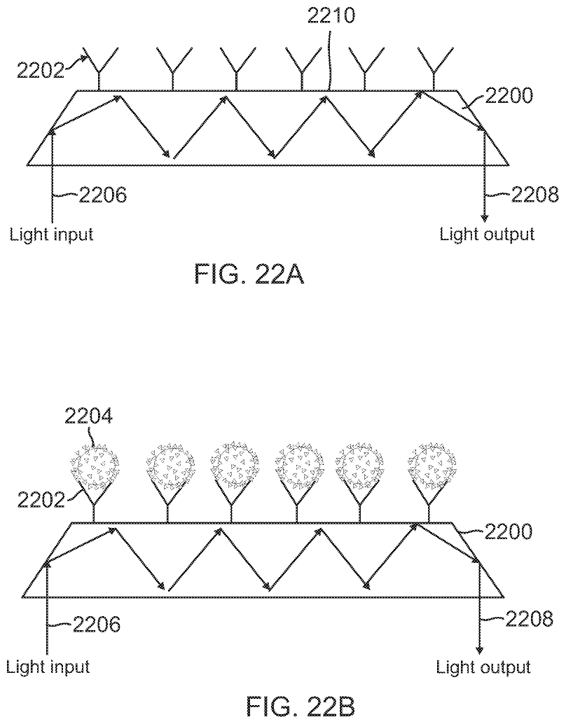

FIGS. 22A and 22B are diagrams illustrating an example of a functionalized ATR element 2200 according to some aspects. For example, the ATR element 2200 (ATR crystal) may include a functionalized top surface 2210 for a biological sample to be detected. The functionalization implies the creation of receptors 2202 on the surface 2210 of the ATR crystal 2200. In an example operation, as shown in FIG. 22A, the functionalized ATR crystal 2200 is used to conduct a background spectral measurement without applying a sample. For example, input light 2206 may be directed into the ATR crystal 2200 for total internal reflection within the ATR crystal 2200 to produce output light for background measurement. Thereafter, as shown in FIG. 22B, a sample 2204 may be applied and optionally washed. The sample analyte 2204 binds to the receptors 2202, which causes a change in the absorption spectrum of the ATR crystal 2200. A new spectrum can then be measured using the spectrometer. The new spectrum and the background spectrum can be used to calculate the change in the spectrum and this change may be fed to the AI engine. If the analyte for which the receptors 2202 are designed is missing from the sample, there will be no binding, and as such, there will be no change in the signal with respect to the background.

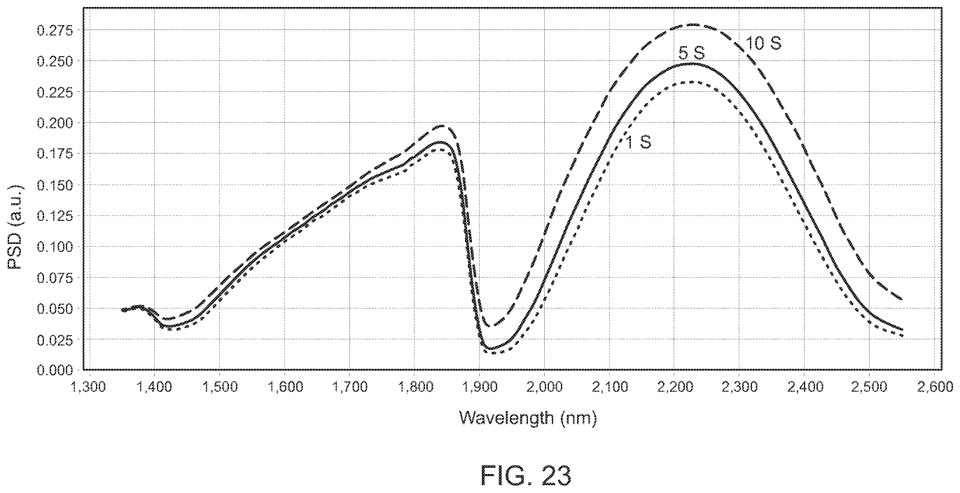

In some examples, the sample can be measured under different excitation conditions to help better distinguish similar samples that are difficult to differentiate by a single measurement. By obtaining multiple spectra of the sample under different excitation conditions, overlapped peaks of the complex spectrum may be spread over multi dimensions. In some examples, the excitation can be in the form of perturbation by an external effect, such as changing the temperature of the sample, changing the power of the light radiation incident on the sample, changing the pressure applied, or other suitable excitation condition. FIG. 23 is a diagram illustrating an example of the effect of different excitations on the measured spectrum. Three different excitations are shown in FIG. 23. Simple division of the spectra obtained at the different excitations can then be used to train the AI engine and also used to produce a result indicative of at least one parameter associated with the sample. Moreover, synchronous and/or asynchronous correlation matrices can be calculated and used to train the AI engine.

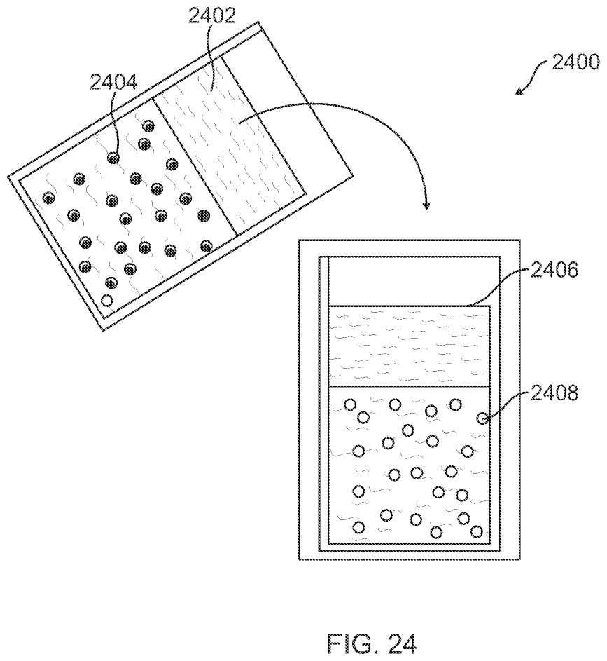

In addition, amplification of the biological signal may be achieved using techniques based on electromagnetic interaction, mechanical techniques, or other techniques. FIG. 24 is a diagram illustrating an example technique for amplifying the signal of a sample according to some aspects. In the example shown in FIG. 24, a sample mixing kit 2400 includes a solution 2402 of quantum dots 2404 with a suitable size and material. The solution 2402 of quantum dots 2404 can be mixed with a media 2406 (e.g., viral transport media) containing a sample 2408 before the spectral measurement. In some examples, the quantum dots 2404 can be in the form of a powder of a colloidal solution. The solvent can be chosen to not affect or interact with the biological sample 2408.

In some examples, the mixed sample (containing the sample 2408 and quantum dots 2404) can be dried before measurements. In some examples, the reaction temperature and drying time may determine the quantum dot size, hence the region of enhancement. As the concentration of the quantum dot increases, the enhancement increases up to a point where the spectrum of the sample 2408 is masked by the QD spectrum. Enhancement factors of up to 10-100× can be reached by adjusting the mixing ratio of the viral transport media 2406 and the quantum dots 2404.

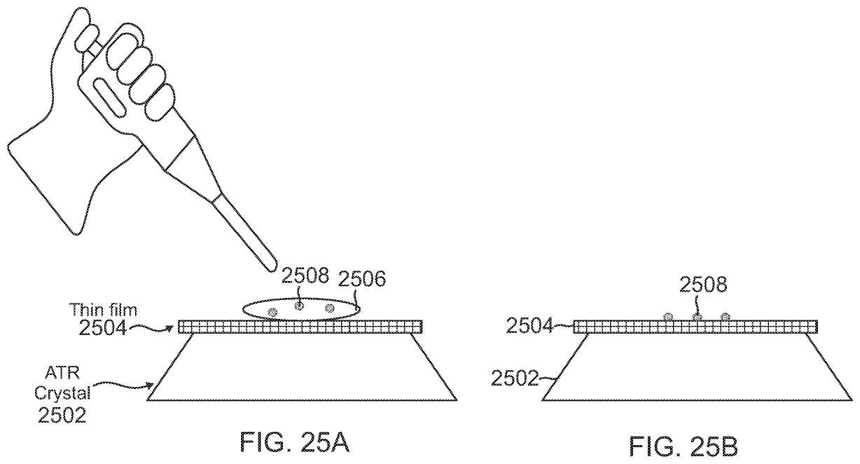

FIGS. 25A and 25B are diagrams illustrating an example of another technique for optical signal enhancement of a sample according to some aspects. In the example shown in FIGS. 25A and 25B, a thin film coated layer 2504 is positioned above an ATR element 2502 (ATR crystal). The thin film coated layer 2504 may be disposable, and as such, correspond to the disposable optical component of the optical element(s) shown in FIG. 3. The thin film coated layer 2504 has a material thickness smaller than the penetration depth of light above the ATR crystal 2502. In some examples, the thin film can be Pentacene, silver, or other suitable material. In examples in which a sample 2508 applied to the thin film coated layer 2504 is contained within a transport media 2506 (as shown in FIG. 25A), the sample 2508 may optionally be dried, as shown in FIG. 25B, to evaporate the media 2506.

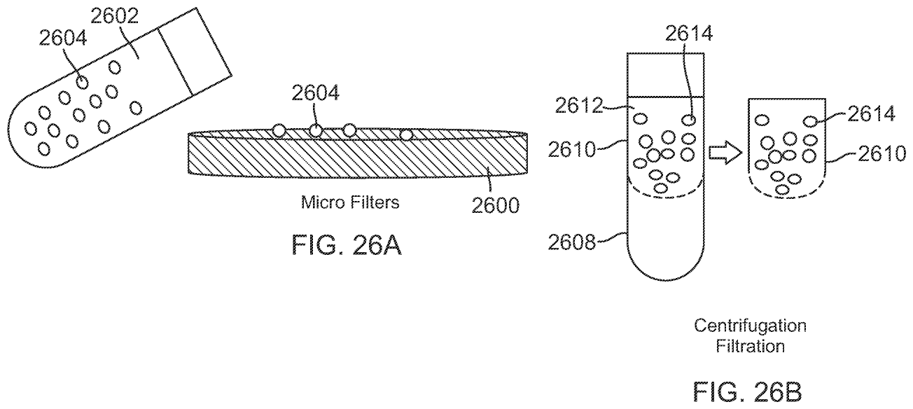

FIGS. 26A and 26B are diagrams illustrating examples of pre-concentration of a sample 2604 according to some aspects. In the example shown in FIG. 26A, a sample 2604 contained within a transport media 2602 may be applied to a disc filter 2600 with small pore size configured to amplify concentration of the sample 2604. For example, the pore size may be on the order of 50-200 nm to ensure that the sample 2604 (e.g., virus) is trapped. The use of such a micro disc filter 2600 enables the sample 2604 (e.g., virus) and media 2602 (e.g., virus transfer media) to be filtered through the disc filter 2600, leaving the virus trapped and accumulated on the top of the filter 2600, resulting in a concentration of the sample 2604 that can increase the sensitivity of the spectrometer.

The disc filter 2600 may be disposable, and as such, correspond to the disposable optical component of the optical element(s) shown in FIG. 3. The disc filter 2600 may be placed in contact with an ATR crystal (not shown) while applying mechanical pressure for intimate contact. In addition, the disc filter 2600 can be measured in transmission mode if infrared light is not blocked.

FIG. 26B illustrates another technique for pre-concentration of a sample 2614. In the technique shown in FIG. 26B, two interdigitated compartments 2608 and 2610 are used, such that the sample 2614 (e.g., virus) is trapped in an inner compartment 2610 and a media 2612 containing the sample 2614 is forced into a lower compartment 2608. A centrifugal force can be applied to separate the sample 2614 from the media 2612.

The following provides an overview of examples of the present disclosure.

Example 1: A spectroscopic analyzer device, comprising: a light source configured to produce incident light; a disposable optical component configured to receive a sample and further configured to receive input light corresponding to the incident light or an interference beam produced based on the incident light, the disposable optical component further configured to produce output light based on light interaction with the sample; a spectrometer configured to receive the incident light from the light source or the output light from the disposable optical component and further configured to produce the interference beam, the interference beam corresponding to the input light or being produced based on the output light; a detector configured to obtain a spectrum of the sample based on the interference beam or the output light; and an artificial intelligence (AI) engine configured to receive the spectrum and to generate a result indicative of at least one parameter associated with the sample.

Example 2: The spectroscopic analyzer device of example 1, wherein the spectrometer comprises a micro-electro-mechanical systems (MEMS) interference device.

Example 3: The spectroscopic analyzer device of example 1 or 2, wherein the disposable optical component comprises an attenuated total internal reflection (ATR) element and the light interaction occurs at an interface between the ATR element and the sample, and further comprising: a first optical coupling element configured to couple the input light into the ATR element; and a second optical coupling element configured to couple the output light out of the ATR element.

Example 4: The spectroscopic analyzer device of example 3, wherein: the ATR element comprises a first surface forming the interface with the sample and a second surface opposite the first surface, and multiple total internal reflections of the input light occur between the first surface and the second surface.

Example 5: The spectroscopic analyzer device of example 4, wherein the first optical element comprises a first lens configured to couple the input light into the ATR element via the second surface and the second optical element comprises a second lens configured to couple the output light from the ATR element via the second surface.

Example 6: The spectroscopic analyzer device of example 4, wherein the first optical element comprises a first lens configured to couple the input light into the ATR element via a first side surface of the ATR element and the second optical element comprises a second lens configured to couple the output light from the ATR element via a second side surface of the ATR element opposite the first side surface, the first side surface having a first face angle configured to receive the input light normal to the first side surface and the second side surface having a second face angle configured to output the output light normal to the second side surface.

Example 7: The spectroscopic analyzer device of example 3, wherein: the ATR element comprises a prism shape having a top surface in contact with the sample, a first side surface, and a second side surface, each of the first side surface and the second side surface having a respective 45 degree face angle, the first optical coupling element comprises a first off-axis reflector configured to receive the input light from a light source and to reflect the input light into the ATR element via the first side surface thereof to produce a single total internal reflection of the input light at the top surface, and the second optical coupling element comprises a second off-axis reflector configured to receive the output light via the second side surface of the ATR element and to reflect the output light into the spectrometer.

Example 8: The spectroscopic analyzer device of any of example 3, wherein: the ATR element comprises a trapezoidal shape having a top surface in contact with the sample, a bottom surface opposite the top surface, a first side surface, and a second side surface opposite the first side surface, each of the first side surface and the second side surface having a respective 45 degree face angle, the first optical coupling element is configured to couple the input light from a light source through the bottom surface towards the first side surface, the input light being subjected to a first total internal reflection thereof at the first side surface to direct the input light to the top surface, the input light being subjected to a single total internal reflection of the input light at the top surface to produce the output light and direct the output light to the second side surface to subject the output light to a second total internal reflection thereof, and the second optical coupling element is configured to couple the output light into the spectrometer.

Example 9: The spectroscopic analyzer device of example 8, wherein the first optical coupling element comprises a first lens and the second optical coupling element comprises a second lens.

Example 10: The spectroscopic analyzer device of examples 8, wherein the first optical coupling element comprises a first curved portion of the bottom surface and the second optical coupling element comprises a second curved portion of the bottom surface.

Example 11: The spectroscopic analyzer device of any of examples 1 through 10, wherein the spectrometer is configured to receive the output light and to produce the interference beam based on the output light, the spectrometer and the detector being assembled together in a single package, the light source and the single package being assembled on a same electronic board.

Example 12: The spectroscopic analyzer device of example 1 or 2, wherein the spectrometer is configured to receive the incident light and to produce the interference beam corresponding to the input light based on the incident light.

Example 13: The spectroscopic analyzer device of example 12, wherein the spectrometer comprises a plurality of interferometers, each configured to receive the incident light and to produce a respective interference beam collectively forming the input light.

Example 14: The spectroscopic analyzer device of any of examples 1, 2, or 11 through 13, further comprising: an attenuated total internal reflection (ATR) element, wherein the disposable optical component comprises a cover slip positioned on the ATR element and configured to receive the sample, the cover slip being inverted to position the sample adjacent the ATR element, the sample being a dried sample, and further comprising: a clamp configured to secure the cover slip.

Example 15: The spectroscopic analyzer device of any of examples 1 through 14, further comprising: a drying agent configured to dry the sample, the detector configured to detect a plurality of spectra of the sample during drying of the sample, the plurality of spectra being input to the AI engine to train the AI engine or produce the result.

Example 16: The spectroscopic analyzer device of any of examples 1, 2, 11 through 13, or 15, wherein the disposable optical component comprises an attenuated total internal reflection (ATR) element, and further comprising: a top cover comprising self-alignment grooves facilitating insertion and removal of the ATR element.

Example 17: The spectroscopic analyzer device of any of examples 1, 2, 11 through 13, or 15, further comprising: an attenuated total internal reflection (ATR) element, wherein the disposable optical component comprise a removable slab positioned above the ATR element and configured to receive the sample.

Example 18: The spectroscopic analyzer device of any of examples 1 through 11 or 14 through 17, wherein the light source comprises: a first light source configured to produce the input light in an attenuated total internal reflection (ATR) mode, the spectrometer being configured to receive the output light and to produce the interference beam based on the output light in the ATR mode; and a second light source configured to produce additional light and to direct the additional input light through the sample and the ATR element in a transmission mode, the spectrometer being further configured to receive additional output light from the ATR element and to produce the interference beam based on the additional output light in the transmission mode.

Example 19: The spectroscopic analyzer device of example 18, wherein the spectrometer comprises a first spectrometer configured to operate in the ATR mode and a second spectrometer configured to operate in the transmission mode.

Example 20: The spectroscopic analyzer device of example 19, further comprising: a sample slide comprising a fluidic splitter, the fluidic splitter comprising an injection cavity configured to receive the sample, a first micro-fluidic channel, a second micro-fluidic channel, an output hole and an output cavity, the fluidic splitter configured to split the sample into a first sample portion and a second sample portion using the first micro-fluidic channel and the second micro-fluidic channel, the fluidic splitter further configured to direct the first sample portion to the output hole via the first micro-fluidic channel for sample measurement in the ATR mode and to direct the second sample portion to the output cavity via the second micro-fluidic channel for sample measurement in the transmission mode.

Example 21: The spectroscopic analyzer device of any of examples 1 through 20, wherein the disposable optical component comprises a functionalized attenuated total internal reflection (ATR) crystal comprising receptors configured to bind with the sample.

Example 22: The spectroscopic analyzer device of example 21, wherein the detector is further configured to detect a background spectrum without the sample on the functionalized ATR crystal, and wherein the AI engine is configured to receive a change between the spectrum and the background spectrum to produce the result.

Example 23: The spectroscopic analyzer device of any of examples 1 through 22, wherein the detector is configured to detect additional spectra of the sample under different excitation conditions and to provide the additional spectra to the AI engine to train the AI engine.

Example 24: The spectroscopic analyzer device of any of examples 1 through 23, further comprising: a sample mixing kit configured to mix the sample with quantum dots, wherein the spectrum of the sample comprises a spectrum of the sample mixed with the quantum dots.

Example 25: The spectroscopic analyzer device of any of examples 1, 2, 11 through 13, 15, 18, 19, 23, or 24 further comprising: an attenuated total internal reflection (ATR) element, wherein the disposable optical element comprises a thin film coated layer above the ATR element configured to receive the sample.

Example 26: The spectroscopic analyzer device of any of examples 1, 2, 11 through 13, 15, 18, 19, 23, or 24 wherein the sample is contained within a media, and further comprising: an attenuated total internal reflection (ATR) element, wherein the disposable optical component comprises a disc filter comprising a pore size configured to amplify concentration of the sample, the disc filter being above the ATR element.

Example 27: The spectroscopic analyzer device of any of examples 1, 2, 11 through 13, 15, 23 or 24, further comprising: a multi-pass cell comprising at least one reflecting element and configured to receive the input light and to produce the output light based on multiple reflections of the input light interacting with the sample, wherein the disposable optical component is within the multi-pass cell.

Example 28: The spectroscopic analyzer device of example 27, wherein the disposable optical component comprises a reflecting element of the at least one reflecting element.

Example 29: The spectroscopic analyzer device of example 27, wherein the disposable optical component comprises a transparent optical window positioned in a light path within the multi-pass cell having a minimized light spot size.

Example 30: The spectroscopic analyzer device of any of examples 27 through 29, wherein the multi-pass cell comprises a circular cell, a white cell, or a heriot cell.

Example 31: The spectroscopic analyzer device of any of examples 1 through 30, further comprising: a twin sample collection device comprising a first part configured to apply the sample to the disposable optical component and a second part configured to enable referencing of the sample to produce a reference result, wherein the AI engine is configured to receive both the spectrum and the reference result.

Example 32: The spectroscopic analyzer device of any of examples 1 through 31, wherein the AI engine is calibrated using a set of clinical samples and a set of laboratory prepared samples using virus stocking and titration.

Within the present disclosure, the word “exemplary” is used to mean “serving as an example, instance, or illustration.” Any implementation or aspect described herein as “exemplary” is not necessarily to be construed as preferred or advantageous over other aspects of the disclosure. Likewise, the term “aspects” does not require that all aspects of the disclosure include the discussed feature, advantage or mode of operation. The term “coupled” is used herein to refer to the direct or indirect coupling between two objects. For example, if object A physically touches object B, and object B touches object C, then objects A and C may still be considered coupled to one another—even if they do not directly physically touch each other. For instance, a first object may be coupled to a second object even though the first object is never directly physically in contact with the second object. The terms “circuit” and “circuitry” are used broadly, and intended to include both hardware implementations of electrical devices and conductors that, when connected and configured, enable the performance of the functions described in the present disclosure, without limitation as to the type of electronic circuits, as well as software implementations of information and instructions that, when executed by a processor, enable the performance of the functions described in the present disclosure.

One or more of the components, steps, features and/or functions illustrated in FIGS. 1-26B may be rearranged and/or combined into a single component, step, feature or function or embodied in several components, steps, or functions. Additional elements, components, steps, and/or functions may also be added without departing from novel features disclosed herein. The apparatus, devices, and/or components illustrated in FIGS. 1-26B may be configured to perform one or more of the methods, features, or steps described herein. The novel algorithms described herein may also be efficiently implemented in software and/or embedded in hardware.

It is to be understood that the specific order or hierarchy of steps in the methods disclosed is an illustration of exemplary processes. Based upon design preferences, it is understood that the specific order or hierarchy of steps in the methods may be rearranged. The accompanying method claims present elements of the various steps in a sample order, and are not meant to be limited to the specific order or hierarchy presented unless specifically recited therein.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language of the claims, wherein reference to an element in the singular is not intended to mean “one and only one” unless specifically so stated, but rather “one or more.” Unless specifically stated otherwise, the term “some” refers to one or more. A phrase referring to “at least one of” a list of items refers to any combination of those items, including single members. As an example, “at least one of: a, b, or c” is intended to cover: a; b; c; a and b; a and c; b and c; and a, b and c. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

What is claimed is:

1. A spectroscopic analyzer device, comprising:
a light source configured to produce incident light;
a disposable optical component configured to receive a sample and further configured to receive input light corresponding to the incident light or an interference beam produced based on the incident light, the disposable optical component further configured to produce output light based on light interaction with the sample;
a spectrometer configured to receive the incident light from the light source or the output light from the disposable optical component and further configured to produce the interference beam, the interference beam corresponding to the input light or being produced based on the output light;
a detector configured to obtain a spectrum of the sample based on the interference beam or the output light; and
an artificial intelligence (AI) engine configured to receive the spectrum and to generate a result indicative of at least one parameter associated with the sample.

2. The spectroscopic analyzer device of claim 1, wherein the spectrometer comprises a micro-electro-mechanical systems (MEMS) interference device.

3. The spectroscopic analyzer device of claim 1, wherein the disposable optical component comprises an attenuated total internal reflection (ATR) element and the light interaction occurs at an interface between the ATR element and the sample, and further comprising:
a first optical coupling element configured to couple the input light into the ATR element; and
a second optical coupling element configured to couple the output light out of the ATR element.

4. The spectroscopic analyzer device of claim 3, wherein:
the ATR element comprises a first surface forming the interface with the sample and a second surface opposite the first surface, and
multiple total internal reflections of the input light occur between the first surface and the second surface.

5. The spectroscopic analyzer device of claim 4, wherein the first optical coupling element comprises a first lens configured to couple the input light into the ATR element via the second surface and the second optical coupling element comprises a second lens configured to couple the output light from the ATR element via the second surface.

6. The spectroscopic analyzer device of claim 4, wherein the first optical coupling element comprises a first lens configured to couple the input light into the ATR element via a first side surface of the ATR element and the second optical coupling element comprises a second lens configured to couple the output light from the ATR element via a second side surface of the ATR element opposite the first side surface, the first side surface having a first face angle configured to receive the input light normal to the first side surface and the second side surface having a second face angle configured to output the output light normal to the second side surface.

7. The spectroscopic analyzer device of claim 3, wherein:
the ATR element comprises a prism shape having a top surface in contact with the sample, a first side surface, and a second side surface, each of the first side surface and the second side surface having a respective 45 degree face angle,
the first optical coupling element comprises a first off-axis reflector configured to receive the input light from the light source and to reflect the input light into the ATR element via the first side surface thereof to produce a single total internal reflection of the input light at the top surface, and
the second optical coupling element comprises a second off-axis reflector configured to receive the output light via the second side surface of the ATR element and to reflect the output light into the spectrometer.

8. The spectroscopic analyzer device of claim 3, wherein:
the ATR element comprises a trapezoidal shape having a top surface in contact with the sample, a bottom surface opposite the top surface, a first side surface, and a second side surface opposite the first side surface, each of the first side surface and the second side surface having a respective 45 degree face angle,
the first optical coupling element is configured to couple the input light from the light source through the bottom surface towards the first side surface, the input light being subjected to a first total internal reflection thereof at the first side surface to direct the input light to the top surface, the input light being subjected to a single total internal reflection of the input light at the top surface to produce the output light and direct the output light to the second side surface to subject the output light to a second total internal reflection thereof, and
the second optical coupling element is configured to couple the output light into the spectrometer.

9. The spectroscopic analyzer device of claim 8, wherein the first optical coupling element comprises a first lens and the second optical coupling element comprises a second lens.

10. The spectroscopic analyzer device of claim 8, wherein the first optical coupling element comprises a first curved portion of the bottom surface and the second optical coupling element comprises a second curved portion of the bottom surface.

11. The spectroscopic analyzer device of claim 1, wherein the spectrometer is configured to receive the output light and to produce the interference beam based on the output light, the spectrometer and the detector being assembled together in a single package, the light source and the single package being assembled on a same electronic board.

12. The spectroscopic analyzer device of claim 1, wherein the spectrometer is configured to receive the incident light and to produce the interference beam corresponding to the input light based on the incident light.

13. The spectroscopic analyzer device of claim 12, wherein the spectrometer comprises a plurality of interferometers, each configured to receive the incident light and to produce a respective interference beam collectively forming the input light.

14. The spectroscopic analyzer device of claim 1, further comprising:
an attenuated total internal reflection (ATR) element, wherein the disposable optical component comprises a cover slip positioned on the ATR element and configured to receive the sample, the cover slip being inverted to position the sample adjacent the ATR element, the sample being a dried sample, and further comprising:
a clamp configured to secure the cover slip.

15. The spectroscopic analyzer device of claim 1, further comprising:
a drying agent configured to dry the sample, the detector configured to detect a plurality of spectra of the sample during drying of the sample, the plurality of spectra being input to the AI engine to train the AI engine or produce the result.

16. The spectroscopic analyzer device of claim 1, wherein the disposable optical component comprises an attenuated total internal reflection (ATR) element, and further comprising:
a top cover comprising self-alignment grooves facilitating insertion and removal of the ATR element.

17. The spectroscopic analyzer device of claim 1, further comprising:
an attenuated total internal reflection (ATR) element, wherein the disposable optical component comprise a removable slab positioned above the ATR element and configured to receive the sample.

18. The spectroscopic analyzer device of claim 1, wherein the light source comprises:
a first light source configured to produce the input light in an attenuated total internal reflection (ATR) mode, the spectrometer being configured to receive the output light and to produce the interference beam based on the output light in the ATR mode; and
a second light source configured to produce additional input light and to direct the additional input light through the sample and the ATR element in a transmission mode, the spectrometer being further configured to receive additional output light from the ATR element and to produce the interference beam based on the additional output light in the transmission mode.

19. The spectroscopic analyzer device of claim 18, wherein the spectrometer comprises a first spectrometer configured to operate in the ATR mode and a second spectrometer configured to operate in the transmission mode.

20. The spectroscopic analyzer device of claim 19, further comprising:
a sample slide comprising a fluidic splitter, the fluidic splitter comprising an injection cavity configured to receive the sample, a first micro-fluidic channel, a second micro-fluidic channel, an output hole and an output cavity, the fluidic splitter configured to split the sample into a first sample portion and a second sample portion using the first micro-fluidic channel and the second micro-fluidic channel, the fluidic splitter further configured to direct the first sample portion to the output hole via the first micro-fluidic channel for sample measurement in the ATR mode and to direct the second sample portion to the output cavity via the second micro-fluidic channel for sample measurement in the transmission mode.

21. The spectroscopic analyzer device of claim 1, wherein the disposable optical component comprises a functionalized attenuated total internal reflection (ATR) crystal comprising receptors configured to bind with the sample.

22. The spectroscopic analyzer device of claim 21, wherein the detector is further configured to detect a background spectrum without the sample on the functionalized ATR crystal, and wherein the AI engine is configured to receive a change between the spectrum and the background spectrum to produce the result.

23. The spectroscopic analyzer device of claim 1, wherein the detector is configured to detect additional spectra of the sample under different excitation conditions and to provide the additional spectra to the AI engine to train the AI engine.

24. The spectroscopic analyzer device of claim 1, further comprising:
a sample mixing kit configured to mix the sample with quantum dots, wherein the spectrum of the sample comprises a spectrum of the sample mixed with the quantum dots.

25. The spectroscopic analyzer device of claim 1, further comprising:
an attenuated total internal reflection (ATR) element, wherein the disposable optical component comprises a thin film coated layer above the ATR element configured to receive the sample.

26. The spectroscopic analyzer device of claim 1, wherein the sample is contained within a media, and further comprising:
an attenuated total internal reflection (ATR) element, wherein the disposable optical component comprises a disc filter comprising a pore size configured to amplify concentration of the sample, the disc filter being above the ATR element.

27. The spectroscopic analyzer device 1, further comprising:
a multi-pass cell comprising at least one reflecting element and configured to receive the input light and to produce the output light based on multiple reflections of the input light interacting with the sample, wherein the disposable optical component is within the multi-pass cell.

28. The spectroscopic analyzer device of claim 27, wherein the disposable optical component comprises a reflecting element of the at least one reflecting element.

29. The spectroscopic analyzer device of claim 27, wherein the disposable optical component comprises a transparent optical window positioned in a light path within the multi-pass cell having a minimized light spot size.

30. The spectroscopic analyzer device of claim 27, wherein the multi-pass cell comprises a circular cell, a white cell, or a heriot cell.

31. The spectroscopic analyzer device of claim 1, further comprising:
a twin sample collection device comprising a first part configured to apply the sample to the disposable optical component and a second part configured to enable referencing of the sample to produce a reference result, wherein the AI engine is configured to receive both the spectrum and the reference result.

32. The spectroscopic analyzer device of claim 1, wherein the AI engine is calibrated using a set of clinical samples and a set of laboratory prepared samples using virus stocking and titration.
